(19) United States
(12) Patent Application Publication
(10) Pub. No.: US 2002/0133149 A1
(43) Pub. Date: Sep. 19, 2002

(54) Electrosurgical systems and methods for hair transplantation and epilation

(75) Inventor: Andre P. Bessette, San Francisco, CA (US)

Correspondence Address: ARTHROCARE CORPORATION, 680 VAQUEROS AVENUE, SUNNYVALE, CA, 94085-3523, US

(73) Assignee: ArthroCare Corporation, Sunnyvale, CA

(21) Appl. No.: 10/097,763

(22) Filed: Mar. 13, 2002

Related U.S. Application Data
(60) Provisional application No. 60/276,863, filed on Mar. 17, 2001.

(51) Int. Cl. A61B 18/18

(52) U.S. Cl. 606/41

(57) ABSTRACT

Electrosurgical systems, apparatus, and methods for the controlled ablation, cutting, forming voids, or otherwise treating skin tissue. An electrosurgical apparatus of the invention includes a treatment assembly including a plurality of treatment units, each treatment unit having an active electrode terminal adapted for the controlled, localized ablation of skin tissue. A method of performing an autologous hair transplant procedure includes electrosurgically forming a plurality of graft sockets in a recipient region of skin tissue. Each of the plurality of graft sockets comprises a void in the skin tissue, formed by positioning a treatment unit in at least close proximity to the skin surface in the recipient region, and thereafter applying a high frequency voltage between the active electrode terminal and a return electrode. Apparatus and methods for epilating the skin are also disclosed.

### RELATED APPLICATIONS

The present invention claims priority from U.S. Provisional Patent Application No. 60/276,863 filed Mar. 17, 2001.

### BACKGROUND OF THE INVENTION

The present invention relates generally to the field of electrosurgery, and more particularly to surgical devices and methods which employ high frequency electrical energy to treat a patient's skin and subcutaneous tissue, including skin resurfacing procedures, the removal of pigmentations, vascular lesions, scars and tattoos, hair removal and/or hair transplant procedures, treatment of skin cancer, skin rejuvenation (e.g., wrinkle removal), liposuction, blepharoplasty, and the like.

In early dermatology procedures, cosmetic surgeons often employed chemical peels and/or dermabrasion techniques to remove outer layers of the patient's skin to rejuvenate wrinkled skin or to remove skins disorders, such as acne, lesions, early skin cancer, etc. These dermabrasion and chemical procedures, however, are difficult to control, requiring great surgical skill. In addition, these somewhat inelegant techniques often cause excessive bleeding, collateral tissue damage and patient discomfort.

In an effort to overcome some of the limitations of dermabrasion and chemical peels, lasers have been developed for use in various dermatological procedures, e.g., for cosmetic surgery. Lasers have improved the accuracy of skin resurfacing procedures, and they have reduced collateral damage to the tissue surrounding and underlying the treatment site. In laser dermatology applications, a hand-piece is typically used to guide the output of a laser to the patient's skin, and to form a laser spot of a desired size on the region of the skin to be treated. The hand-piece is typically attached to one end of an articulated arm which transmits the output of a medical laser (such as CO₂ or Er: YAG lasers) to the hand-piece and allows the hand-piece a wide range of motion. Lasers may also have applications in hair removal and hair transplantation procedures.

Although initially promising, lasers suffer from a number of drawbacks in dermatology procedures. For example, laser equipment can be very expensive because of the costs associated with the laser light sources. Moreover, those lasers which permit acceptable depths of necrosis (such as excimer lasers, erbium:YAG lasers, and the like) provide a very low volumetric ablation rate, requiring numerous passes over the same treatment area, which results in longer procedural times. In addition, erbium:YAG lasers generally do not provide effective hemostasis during the procedure, resulting in excessive bleeding which disrupts the surgeon's view of the treatment site. The CO₂ lasers provide a higher rate of ablation and an increased depth of tissue necrosis than their erbium: YAG counterparts. On the other hand, CO₂ lasers often create significant residual thermal injury to tissue at and surrounding the treatment site, which prolongs the healing period for the patient. In addition, CO₂ lasers are associated with much pain and, therefore, require a lot of anesthesia, which increases the cost and length of the procedure.

In the treatment of vascular skin lesions, lasers have been used to irradiate the surface of the skin. The laser energy penetrates through the skin and is absorbed in the blood, which coagulates and collapses the blood vessel. Unfortunately, there are also problems associated with the use of lasers in these procedures. For example, although most of the laser energy passes through the tissue to the vessel, scattering and absorption of the light take place in the tissue. This absorption can cause significant changes in skin coloration and even scarring.

Monopolar electrosurgical instruments have been used for various dermatogical procedures. For example, Conmed Corporation manufacturers a monopolar device, termed the Hyfrecator™ having a single active electrode at the tip of an electrosurgical probe. In these procedures, at least a portion of the skin abnormality is typically removed with a scalpel, and thereafter, a low voltage is applied to the active electrode in contact with the target tissue to deliver electric current through the tissue and the patient to a dispersive pad or return electrode. The voltage desiccates the remaining abnormal tissue, and coagulates severed blood vessels at the target site. The remaining tissue is then removed with a sponge or similar material. The voltage generally must be low enough to prevent charring and potential scarring of the underlying dermis.

Electrosurgical instruments that deliver RF energy have been used to remove or otherwise treat skin tissue in various procedures, since they generally reduce patient bleeding associated with tissue cutting operations and improve the surgeon's visibility. For example, in blepharoplasty procedures (for treating baggy eyelids), monopolar RF devices, e.g., the Colorado Needle™, are frequently used to create the necessary incisions in the patient's eyelids. However, many prior art electrosurgical devices and procedures, suffer from a number of disadvantages. For example, conventional electrosurgical cutting devices typically operate by creating a voltage difference between the active electrode and the target tissue, causing an electrical arc to form across the physical gap between the electrode and the tissue. At the point of contact of the electric arcs with the tissue, rapid tissue heating occurs due to high current density between the electrode and the tissue. This high current density causes cellular fluids to rapidly vaporize into steam, thereby producing a “cutting effect” along the pathway of localized tissue heating. This cutting effect generally results in the production of smoke, or an electrosurgical plume, which can spread bacterial or viral particles from the tissue to the surgical team or to other portions of the patient's body. In addition, the tissue is parted along the pathway of evaporated cellular fluid, inducing undesirable collateral tissue damage in regions surrounding the target tissue site. Bipolar electrosurgical instruments offer a number of advantages, as compared with monopolar devices. For example, in the case of bipolar electrosurgical instruments, the return current path does not flow through the patient's body beyond the immediate site of application of the instrument.

Thus, there is a need for bipolar electrosurgical instruments adapted for performing dermatological procedures, including hair removal (epilation), and hair transplant procedures. Hair loss (alopecia) is an extremely common condition in humans, as well as in other mammals. Alopecia may be naturally occurring (primary alopecia), or it may be induced by chemical or physical agents (secondary alopecia). Hair loss may also result from specific disease states, such as mange, or formation of scar tissue, and with increasing age. Many healthy adult male humans suffer from hair loss. Adult female humans also commonly experience excessive hair loss. Alopecia is also frequently observed in both pre- and post-pubertal patients as a side effect of anticancer chemotherapy. In some cancer patients, the likelihood of chemotherapy-induced alopecia may lead to a refusal to accept treatment.

Despite the widespread occurrence of alopecia, and extensive research efforts to find suitable remedies, effective clinical treatments are lacking. Currently the most effective treatment for alopecia is hair transplant surgery. Conventional hair transplant procedures have employed mechanical surgical instruments, such as scalpels, multi-blade devices, and punches for making incisions in the scalp. More recently, lasers have been used in hair transplant procedures. Lasers suffer from a number of disadvantages, as outlined hereinabove. Thus, there is a need for bipolar electrosurgical instruments adapted for performing various steps involved in hair transplant procedures.

On the other hand, millions of men and women routinely attempt to remove unwanted body hair. Currently, innumerable products are marketed for hair removal. Such products include razors, hair-removal wax, chemical depilatories, and tweezers. While these products may be partially effective for the temporary removal of hair, none of these products permanently removes body hair.

One prior art process for removing unwanted body hair is electrolysis. In conventional electrolysis procedures for hair removal, an electrologist inserts a thin probe into a hair follicle adjacent to a hair root. Heat is delivered to the probe by a device known as an epilator. The nourishing cells beneath the root of the hair are destroyed permanently by the heat. Unfortunately, electrolysis is often tedious, time consuming, and often causes excessive bleeding, collateral tissue damage, and patient discomfort.

In an effort to overcome some of the limitations of electrolysis, lasers have been used in hair removal procedures. Lasers have been found to provide reduced collateral damage to the tissue surrounding and underlying the treatment site, as compared with conventional electrolysis. In laser hair removal applications, a hand-piece is typically used to guide the output of a laser to the patient's skin, and to form a laser spot of a desired size on the region of the skin which is to be treated.

However, as described hereinabove, dermatological procedures using lasers often suffer from a number of drawbacks, such as excessive pain, the need for anesthesia, and extended recovery time. Moreover, some laser procedures merely induce a growth delay in a treated hair follicle, instead of causing permanent hair removal. Thus, there is a need for improved systems, apparatus, and methods for removing unwanted hair.

### SUMMARY OF THE INVENTION

The present invention provides systems, apparatus, and methods for selectively applying electrical energy to the epidermis, dermis, or underlying subcutaneous tissue of a patient. The systems and methods of the present invention are particularly useful for creating precise incisions, indentations, voids, or sockets in the skin, with minimal pain and postoperative scarring.

In one aspect of the invention, a method for removing fatty tissue underlying a patient's epidermis is disclosed (e.g., blepharoplasty, brow lifts, eyelid shortening procedures, and the like). This method includes positioning one or more active electrode(s) and one or more return electrode(s) in close proximity to a target site on an external body surface of the patient. A high frequency voltage difference is applied between the active and return electrode(s), and the active electrode(s) are translated across the external body surface to create an incision therein. The bipolar configuration of the present invention controls the flow of current to the immediate region around the distal end of the probe, which minimizes tissue necrosis and the conduction of current through the patient. The residual heat from the electrical energy also provides simultaneous hemostasis of severed blood vessels, which increases visualization and improves recovery time for the patient. The techniques of the present invention produce significantly less thermal energy than many conventional techniques, such as lasers and conventional RF devices, which reduces collateral tissue damage and minimizes pain and postoperative scarring.

In some procedures, such as blepharoplasty, the incision is selected to allow a portion of external skin to be removed (i.e., folded over or removed entirely) to expose the underlying tissue, such as the orbital septum in blepharoplasty procedures. Depending on the specific procedure, the fatty portions of the underlying tissue are then removed either with conventional tools, such as a scalpel, or with the electrosurgical instruments described herein. In the latter embodiment, a sufficient high frequency voltage is applied between the active and return electrode(s) to volumetrically remove this fatty tissue either in situ, or by breaking up the tissue sufficiently to enable removal by suction pressure (e.g., liposuction). In this latter case, the tissue may be removed by an electrosurgical probe having an aspiration lumen and/or an aspiration electrode to prevent clogging of the lumen. A more complete description of such a device can be found in Ser. No. 09/010,382, filed Jan. 21, 1998 (attorney docket A-6), previously incorporated herein by reference.

The return electrode(s) are preferably spaced from the active electrode(s) a sufficient distance to prevent arcing therebetween at the voltages suitable for tissue removal, and to prevent contact of the return electrode(s) with the tissue. The current flow path between the active and return electrodes may be generated by directing an electrically conductive fluid along a fluid path past the return electrode and to the target site, or by locating a viscous electrically conductive fluid, such as a gel, at the target site, and submersing the active and return electrode(s) within the conductive gel. The electrically conductive fluid is selected to have sufficient electrical conductivity to allow current to pass therethrough from the active to the return electrode, and such that the fluid ionizes into a plasma when subjected to sufficient electrical energy, as discussed below. In the exemplary embodiment, the conductive fluid is isotonic saline, although other fluids may be selected.

In an exemplary embodiment, the incision is created by removing tissue with molecular dissociation or disintegration processes. Conventional electrosurgery cuts through tissue by rapidly heating the tissue until cellular fluids explode, producing a cutting effect along the pathway of localized heating. The present invention volumetrically removes the tissue in a cool ablation process that minimizes thermal damage to surrounding tissue. In these processes, the high frequency voltage applied to the active electrode(s) is sufficient to vaporize an electrically conductive fluid (e.g., gel or saline) between the electrode(s) and the tissue. Within the vaporized fluid, a ionized plasma is formed and charged particles (e.g., electrons) are accelerated towards the tissue to cause the molecular breakdown or disintegration of several cell layers of the tissue. This molecular dissociation is accompanied by the volumetric removal of the tissue. This process can be precisely controlled to effect the volumetric removal of tissue as thin as 10 to 50 microns with minimal heating of, or damage to, surrounding or underlying tissue structures. A more complete description of this phenomenon is described in commonly assigned U.S. Pat. No. 5,683,366, the complete disclosure of which is incorporated herein by reference.

The present invention offers a number of advantages over current RF and laser techniques for creating incisions in skin. The ability to precisely control the volumetric removal of tissue results in a field of tissue cutting or removal that is very defined, consistent, and predictable. Controlling the depth of tissue removal allows the physician to form a precise incision through the skin tissue. This precise heating also helps to minimize, or completely eliminate, damage to healthy tissue structures or nerves that are often adjacent to the target tissue. In addition, small blood vessels within the skin tissue are simultaneously cauterized and sealed as the tissue is removed to continuously maintain hemostasis during the procedure. This increases the surgeon's field of view, and shortens the length of the procedure. Moreover, since the present invention allows for the use of electrically conductive fluid (contrary to prior art bipolar and monopolar electrosurgery techniques), isotonic saline may be used during the procedure. Isotonic saline is the preferred medium for irrigation because it has the same osmotic pressure as intracellular fluid, suitable electrical conductivity, and is transparent to visible light.

Apparatus according to the present invention generally include an electrosurgical probe or hand-piece having a shaft or handle with proximal and distal ends and an electrode assembly at the distal end. The apparatus will preferably further include a fluid delivery element for delivering electrically conductive fluid to the electrode terminal(s) and the target site. The fluid delivery element may be located on the probe, e.g., a fluid lumen or tube, or it may be part of a separate instrument. Alternatively, an electrically conductive gel or spray, such as a saline electrolyte or other conductive gel, may be applied to the target site. In this embodiment, the apparatus may not have a fluid delivery element. In both embodiments, the electrically conductive fluid will preferably generate a current flow path from the electrode terminals to one or more return electrode(s). In an exemplary embodiment, the return electrode is located on the probe, and spaced a sufficient distance from the electrode terminal(s) to substantially avoid or minimize current shorting therebetween, and to shield the return electrode from tissue at the target site.

In a specific configuration, the electrode assembly comprises an active electrode spaced from a return electrode by an electrically insulating electrode support member. In an exemplary embodiment, the active electrode has a substantially elongate shape with a sharp distal end to facilitate the cutting or incision of tissue, usually having a width of about 0.01 mm to 10 mm, preferably about 0.1 to 0.5 mm, and an exposed length of about 5 to 50 mm, preferably about 10 to 30 mm. The return electrode comprises an electrically conductive sleeve surrounding the insulating support member, which extends distally therefrom. Likewise, the support member preferably comprises an insulating sleeve surrounding the active electrode, the latter extending distally from the support member. In an exemplary embodiment, the electrode assembly extends from the distal face of a support member or disposable tip, which may be attached to a handle to facilitate use by the surgeon. The disposable tip typically includes a fluid lumen having a distal opening adjacent to the electrode assembly for delivery of electrically conductive fluid to the target site. In one embodiment, the fluid lumen extends through the disposable tip, although the fluid lumen may also comprises a fluid tube that extends on the exterior of the tip.

In an alternative embodiment, an electrosurgical probe includes one or more electrode terminal(s) designed for cutting tissue; i.e., they typically have a distal edge or point. In this embodiment, the electrode terminal(s) may be aligned with each other to form a linear cutting path through the tissue. The return electrode may comprise a conductive sleeve on the shaft, spaced proximally from the electrode terminal(s). The conductive sleeve may be a separate conductive element attached to an insulating shaft, or an exposed portion of a conductive shaft.

According to one aspect of the present invention, an apparatus for forming voids in the skin includes a handle and a treatment assembly attached to the handle. The handle may include one or more connection blocks for electrically coupling the handle to a high frequency power supply, or to the treatment assembly. The treatment assembly includes at least one treatment unit. Each treatment unit typically includes an active electrode terminal disposed on an electrode support. In one embodiment, the treatment assembly includes a plurality of treatment units, each treatment unit including an active electrode terminal and a proximal return electrode. Alternatively, the treatment assembly may include a plurality of treatment units and a single, common return electrode.

Each treatment unit is adapted for the localized ablation of skin tissue, e.g., to form a void, hole, or socket in the skin. According to one aspect of the invention, the skin tissue is a recipient area for receiving follicular grafts, and each socket is adapted for accommodating a follicular graft. Apparatus of the invention may further include a fluid delivery unit having a fluid delivery lumen and at least one fluid delivery port. Each fluid delivery port delivers electrically conductive fluid to at least one treatment unit. Typically, each fluid delivery port is configured to provide a current flow path from the active electrode of each treatment unit, through the electrically conductive fluid, to the return electrode. The treatment assembly may include a treatment base from which each treatment unit extends distally. The base may be removably attached to the distal end of the handle. The base and/or the distal end of the handle may be beveled. One or both of the handle and the treatment assembly may be disposable or sterilizable. In one embodiment, the treatment assembly is disposable after a single use, while the handle is sterilizable and reusable.

According to another aspect of the invention, there is provided a method for performing an autologous hair transplant procedure, wherein the method includes electrosurgically ablating skin tissue in a recipient area of the skin to form a plurality of graft sockets. Each graft socket may be formed by a treatment unit of an electrosurgical instrument, wherein the instrument includes a handle and a treatment assembly attached to the handle distal end, substantially as described hereinabove. Such an instrument is adapted for the controlled volumetric removal of skin tissue, with minimal or no damage to adjacent non-target tissue. Each graft socket is adapted for receiving a follicular graft, wherein the follicular grafts are dissected from a strip of hair-bearing skin excised from a donor area. In one embodiment, the hair-bearing skin is electrosurgically excised from the donor area in a procedure involving the plasma-induced volumetric removal of skin tissue components. For example, the hair-bearing skin may be excised by the process known as Coblation®, as described in detail herein.

According to another aspect of the invention, there is provided a method for forming a plurality of voids, or graft sockets, in the skin. In one embodiment, such a method involves positioning a treatment assembly of an electrosurgical instrument in at least close proximity to a target site on the external surface of the skin. The treatment assembly includes at least one treatment unit. Each treatment unit includes an active electrode terminal adapted for forming a void, of a pre-determined size range, in the skin at the target site. Upon application of a high frequency voltage to each treatment unit, skin tissue is volumetrically removed in the vicinity of each treatment unit. An electrically conductive fluid may be delivered to each treatment unit prior to application of the high frequency voltage. The treatment units may number up to 16, or more, and may be arranged on the electrode assembly, e.g., in a square configuration, to form an electrode array capable of forming a plurality of regularly spaced voids on the skin at the target site.

In one embodiment, the active electrode terminal of each treatment unit may be brought into contact with the skin surface, so that an electric current passes into the tissue to a particular depth. Alternatively, the active electrode terminal of each treatment unit may be spaced apart from the skin surface, e.g., by a distance in the range of from about 0.05 mm to about 5 mm. In this manner, a gap exists which allows for the continual re-supply of electrically conductive fluid between the distal tip of the active electrode terminal and the target tissue. This situation may promote maintenance of a plasma layer adjacent to the target tissue.

According to another aspect of the invention, there is provided apparatus and methods for epilating a target area of the skin. In one embodiment, a method of removing unwanted hair involves ablating, destroying, or irreversibly damaging at least a portion of a hair follicle so as to prevent future growth of a hair shaft from the hair follicle. Such a method typically involves positioning the distal end of an electrosurgical probe in relation to the skin so that an active electrode of the probe is in at least close proximity to the targeted hair follicle. Typically, the probe includes a solitary shaft bearing a single active electrode, and a return electrode located proximal to the active electrode. The probe may include a connection block or connector unit for electrically coupling the probe to a suitable high frequency power supply. In one embodiment, a stylet-like active electrode of the probe may be inserted to a certain depth within the hair follicle. Alternatively, the active electrode may be spaced apart from the targeted hair follicle by a short distance, e.g., in the range of from about 0.05 mm to about 5 mm, so that a gap exists between the distal tip of the active electrode and the hair follicle.

In one embodiment of a method for epilating the skin, an electrically conductive fluid is delivered to the active electrode in order to promote generation of a plasma layer adjacent to the active electrode, and to provide a current flow path between the active electrode and the return electrode. After, or during, delivery of the electrically conductive fluid, a suitable high frequency voltage is applied between the active and return electrodes to effect the volumetric removal of at least a portion of the targeted hair follicle. The volumetric removal of hair follicle tissue according to the invention generally takes place at a relatively low temperature in the range of from about 45° to 90° C. Alternatively, a portion of a hair follicle, e.g., the hair bulb or the hair papilla may be irreversibly damaged by exposure to a temperature in excess of 90° C., e.g., by the application of RF energy to the hair follicle.

In one embodiment of a method for epilating the skin, the skin of a patient comprises positioning an active electrode terminal of an electrosurgical probe in at least close proximity to a target hair follicle of the skin, and applying a high frequency voltage between the active electrode terminal and a return electrode, wherein application of the high frequency voltage effects the volumetric removal of at least a portion of the tissue of the hair follicle, wherein during said step, the target hair follicle and the surrounding non-target tissue are exposed to a temperature not exceeding about 90° C., and wherein the target hair follicle is destroyed or permanently inactivated.

An electrosurgical apparatus for epilating the skin of a patient comprises a handle including a connection block, the connection block adapted for electrically coupling the apparatus to a high frequency power supply, a solitary shaft attached to the handle, the shaft having a proximal end and a distal end, an electrically insulating electrode support disposed at the shaft distal end, a return electrode disposed on the shaft at a location proximal to the electrode support, a solitary active electrode terminal extending distally from the electrode support, and a fluid delivery unit adapted for delivering an electrically conductive fluid to the shaft distal end such that the electrically conductive fluid provides a current flow path between the active electrode terminal and the return electrode.

In one embodiment, there is provided an electrosurgical apparatus and method for forming a plurality of graft sockets in the skin of a patient, comprising a handle having a proximal end and a distal end and a treatment assembly disposed at the handle distal end. The treatment assembly includes a base and at least one treatment unit affixed to the base and is adapted for removable attachment to the handle. Each treatment unit includes a shaft, an electrically insulating electrode support disposed on the shaft, and an active electrode terminal disposed on the electrode support.

The method of forming a plurality of graft sockets in a recipient area of the skin of a patient comprises positioning an electrosurgical treatment assembly in at least close proximity to the recipient area of the skin, the treatment assembly including a plurality of active electrode terminals and at least one return electrode, and applying a high frequency voltage to each of the plurality of active electrode terminals, the high frequency voltage sufficient to locally ablate skin tissue in the vicinity of each of the plurality of active electrode terminals.

In another embodiment, the treatment assembly comprises a base and a plurality of treatment units affixed to the base, each of the plurality of treatment units including a shaft, an electrically insulating electrode support disposed on the shaft, and an active electrode terminal disposed on the electrode support.

The embodiment for localized ablation of skin tissue comprises a handle and a treatment assembly attached to the handle. The treatment assembly includes a plurality of treatment units which include a shaft having a shaft proximal end and a shaft distal end, an electrode support disposed on the shaft distal end, and an active electrode terminal disposed on the electrode support. The treatment assembly also includes a high frequency power supply electrically coupled to the handle, the high frequency power supply for supplying a high frequency voltage to the treatment assembly.

In one embodiment the sterilizable handle for an electrosurgical instrument comprises a housing, a first proximal connection block for electrically coupling the handle to a high frequency power supply, a second distal connection block disposed within the housing and in electrical communication with the first proximal connection block, and a distal face adapted for detachably accommodating a treatment assembly of the electrosurgical instrument.

According to another aspect of the invention, there is provided methods of performing an autologous hair transplant on a patient, that comprises excising hair-bearing skin from a donor area of the skin of the patient, sectioning the hair-bearing skin to provide a plurality of follicular grafts, electrosurgically ablating skin tissue in a recipient area of the skin of the patient to form a plurality of graft sockets in the recipient area, and inserting one of the plurality of follicular grafts into each of at least a portion of the plurality of graft sockets.

The method of harvesting donor hair from a target site of skin tissue comprises positioning an active electrode terminal in at least close proximity to the target site of the skin tissue, applying a high frequency voltage to the active electrode terminal, the high frequency voltage sufficient to ablate at least a portion of the skin tissue in the vicinity of the active electrode terminal, and excising at least a portion of the skin from the target site.

A further understanding of the nature and advantages of the invention will become apparent by reference to the remaining portions of the specification and drawings.

### BRIEF DESCRIPTION OF THE DRAWINGS

FIG. 1 is a perspective view of an electrosurgical system for treating a patient's skin including an electrosurgical generator and an electrosurgical probe or hand-piece;

FIG. 2 is a perspective view of one embodiment of an electrosurgical probe constructed according to the principles of the present invention;

FIGS. 3A-3C are exploded, isometric views of the probe of FIG. 2;

FIG. 4 is an end view of the distal tip of the probe of FIG. 2, illustrating an electrode support with a plurality of electrode terminals;

FIG. 5 illustrates the electrical connections and the electrode support of the hand-piece of FIG. 2 in greater detail;

FIG. 6 is an end view of an exemplary electrode support comprising a multi-layer wafer with plated conductors for electrodes;

FIGS. 7 and 8 are side views of the electrode support of FIG. 6;

FIGS. 9A, 10A, 11A, 12A, and 13 are side views of the individual wafer layers of the electrode support of FIG. 6;

FIGS. 9B, 10B, 11B, and 12B are cross-sectional views of the individual wafer layers of the electrode support of FIG. 6;

FIGS. 14 and 15 illustrate an alternative multi-layer wafer design according to the present invention;

FIG. 16A illustrates a method for treating the outer layer of a patient's skin in a skin resurfacing procedure, wherein an outer layer of epidermis is removed or ablated and the collagen fibers in the underlying dermis are contracted;

FIG. 16B illustrates a method for treating the outer layer of a patient's skin in a skin resurfacing procedure with an electrosurgical probe having a single, active electrode terminal;

FIG. 17 illustrates a method of skin resurfacing wherein the epidermal layer is separated from the papillary dermis, and then removed by wiping away the separated layer;

FIGS. 18 and 19 illustrate a method for treating a vascular lesion;

FIG. 20 is a cross-sectional view of an alternative electrosurgical probe for applying high frequency voltage to tissue layers on the skin;

FIG. 21 is a graph illustrating the electrical impedance of tissue and isotonic saline with operating frequency;

FIG. 22 illustrates another embodiment of the probe of the present invention, incorporating additional electrodes sized for contraction of tissue;

FIG. 23 illustrates another embodiment of the probe of the present invention, specifically designed for creating incisions in external skin surfaces;

FIGS. 24-26 illustrates a method according to the present invention for removing fatty tissue under the eyelids to treat “baggy eyelids” syndrome;

FIG. 27 is a detailed end view of an electrosurgical probe having an elongate, linear array of electrode terminals suitable for use in surgical cutting;

FIG. 28 is a detailed view of a single electrode terminal having a flattened end at its distal tip;

FIG. 29 is a detailed view of a single electrode terminal having a pointed end at its distal tip;

FIG. 30 is a perspective view of another embodiment of an electrosurgical probe for use in dermatology procedures;

FIG. 31 is a detailed view of the distal portion of yet another electrosurgical probe according to the present invention;

FIG. 32 is a block diagram representing an electrosurgical system, according to another embodiment of the present invention;

FIGS. 33A and 33B each schematically represent an electrosurgical instrument, according to the instant invention;

FIG. 34A schematically represents an electrosurgical instrument including a treatment assembly, according to the instant invention;

FIGS. 34B and 34C show a perspective view, and a side view, respectively, of the treatment assembly of the electrosurgical instrument of FIG. 34A;

FIG. 35A schematically represents an electrosurgical instrument including a beveled treatment assembly having a plurality of treatment units, according to another embodiment of the invention;

FIGS. 35B, 35C, and 35D show a side view, a perspective view, and an end view, respectively, of the treatment assembly of FIG. 35A;

FIG. 36 schematically represents an electrosurgical instrument having a treatment unit arranged at an angle to a beveled distal end portion of the instrument;

FIG. 37A schematically represents formation of a plurality of graft sockets in the scalp of a patient;

FIG. 37B shows a plurality of graft sockets formed at an angle to the surface of the skin of a patient, according to one embodiment of the invention;

FIGS. 38A and 38B each show a side view of a treatment unit of an electrosurgical instrument, according to two different embodiments;

FIGS. 39A and 39B show a side view and an end view, respectively, of an electrosurgical instrument, according to another embodiment of the invention;

FIG. 40 schematically represents a series of steps involved in a method of performing a hair transplant procedure, according to another embodiment of the invention;

FIG. 41 schematically represents a series of steps involved in a method of forming a plurality of voids in the skin of a patient, according to another embodiment of the invention;

FIGS. 42A and 42B each show, in side view, an electrosurgical probe, according to two different embodiments of the invention;

FIG. 43A shows in side view the distal portion of an electrosurgical probe according to another embodiment of the invention;

FIG. 43B is a sectional view taken along the lines 43B-43B of FIG. 43A;

FIG. 43C is an end view of the probe of FIG. 43A;

FIG. 44A represents a side view of a hair follicle and a hair shaft in the skin of a patient;

FIG. 44B shows the distal end of an electrosurgical probe inserted into a hair follicle, according to one embodiment of the invention;

FIG. 44C shows an electrosurgical probe positioned with respect to the surface of the skin, according to another embodiment of the invention; and

FIG. 45 schematically represents a series of steps in a method of epilating the skin of a patient, according to another embodiment of the invention.

### DESCRIPTION OF SPECIFIC EMBODIMENTS

The present invention provides systems and methods for selectively applying electrical energy to a target location within or on a patient's body, particularly including procedures on an external body surface, such as epidermal and dermal tissues in the skin, or the underlying sub-cutaneous tissue. For convenience, the remaining disclosure is directed primarily to skin tissue, including cutting, ablation, incising or removal of tissue in the epidermis and/or dermis, e.g., the removal of pigmentations, vascular lesions (e.g., leg veins), scars, tattoos, etc., and for other surgical procedures on the skin, such as tissue rejuvenation, blepharoplasty, browlifts, liposuction, cosmetic surgery, wrinkle removal, epilation (hair removal), and/or hair transplant procedures. However, it will be appreciated that the systems, apparatus, and methods of the invention may be equally applicable to procedures involving other tissues of the body, including open surgery, arthroscopic surgery, laparoscopic surgery, thoracoscopic surgery, and other endoscopic surgical procedures.

The present invention applies high frequency or radio frequency (RF) electrical energy to one or more electrode terminals adjacent to an external body surface, such as the outer surface of the skin, to remove and/or modify the structure of the skin tissue. Depending on the specific procedure, the present invention may be used to: (1) volumetrically remove tissue (i.e., ablate or effect molecular dissociation of the tissue structure); (2) decouple or separate a first tissue layer from an underlying tissue layer so that the first tissue layer can later be removed; (3) shrink or contract collagen fibers within connective tissue; (4) create precise voids, sockets, holes, or incisions in the skin or external skin surface; (5) coagulate blood vessels underlying the surface of the skin; (6) perform autologous hair transplant procedures; and/or (7) remove unwanted hair from the skin (epilation).

The skin or integumentary system is the largest organ of the human body. It acts as an interface between the internal and external environment, and fulfills thermoregulatory, barrier, and sensory functions, among others. Histologically, three major tissue layers are identified. The uppermost layer, the epidermis, is a relatively thin stratified squamous epithelium which, in some regions, is itself composed of five strata. Subjacent to the epidermis is the dermis, a dense fibroelastic connective tissue stroma. The third layer, lying beneath the dermis, is the subcutaneous layer composed of fatty connective tissue.

There are two types of skin: hair-bearing skin, which covers the vast majority of the body surface; and hairless skin confined to areas such as the palms of the hands, soles of the feet, and mucous membranes. The two skin types are differentiated on the basis of the presence or absence of the pilosebaceous apparatus: the hair follicle and the accompanying sebaceous gland.

In one aspect, the invention is concerned with hair transplant procedures. In another aspect, the invention is concerned with the removal of excess or unwanted hair. Hairs (or pili) are filamentous, keratinized structures derived from the epidermis. Hairs have a number of roles, including thermoregulation, sensory perception, social communication, and protection of the skin and underlying structures. The density of hairs per unit area of skin varies with species, strain, and skin site. For example in humans, it ranges from about 600 cm-² to about 60 cm-², with the highest density being on the face.

Hairs show enormous variation in the length and diameter of the hair shaft: from <1 mm to >1,000 mm in length, and from 0.005 mm to 0.5 mm in diameter. There are also major differences, within a given individual, in the degree of pigmentation. Two broad categories of hairs are recognized: vellus hairs are short and narrow, and are present over most of the body surface; while terminal hairs are longer, thicker, and often heavily pigmented. Terminal hairs include those of the scalp, eyebrows and eyelashes, as well as the post-pubertal hair of the axillae and pubis, and the facial and body hair in many males.

Each hair consists of a shaft and a root. The hair shaft is composed of specialized cells (keratinocytes) containing a particularly strong form of keratin, providing a filament of high tensile strength. The root lies within the hair follicle, which is an invagination of the epidermis. The hair follicle may extend deeply into the hypodermis or may be more superficial in the dermis. Each follicle contains a hair bulb (or bulbus pili). The hair bulb comprises the germinative matrix, a zone of great mitotic activity which generates the hair and its surrounding inner root sheath, and the keratogenous zone, in which cells are keratinized. The germinative matrix consists of a mass of pluripotent cells capping the dermal papilla. Cells arising mitotically from this group move apically, and may differentiate along several different routes. The activity of the hair bulb, and of the whole root complex involves various morphogenetic processes in which different cell shapes, chemical forms of keratin, and cellular migration patterns are produced.

In one method of the present invention, incisions are created in the skin by volumetrically removing or ablating tissue along a cutting path. In this procedure, a high frequency voltage difference is applied between one or more electrode terminal(s) and one or more return electrode(s) to develop high electric field intensities in the vicinity of a target site. The high electric field intensities lead to electric field induced molecular breakdown of target tissue through molecular dissociation (rather than thermal evaporation or carbonization). Applicant believes that the tissue structure is volumetrically removed through molecular disintegration of larger organic molecules into smaller molecules and/or atoms, such as hydrogen, oxides of carbon, hydrocarbons, and nitrogen compounds. This molecular disintegration completely removes the tissue structure, as opposed to dehydrating the tissue material by the removal of water within the cells of the tissue, as is typically the case with electrosurgical desiccation and vaporization.

The high electric field intensities may be generated by applying a high frequency voltage that is sufficient to vaporize an electrically conductive fluid over at least a portion of the electrode terminal(s) in the region between the distal tip of the electrode terminal(s) and the target tissue. The electrically conductive fluid may be a liquid, such as isotonic saline, delivered to the target site, or a viscous fluid, such as a gel, that is located at the target site. In the latter embodiment, the electrode terminal(s) are submersed in the electrically conductive gel during the surgical procedure. Since the vapor layer or vaporized region has a relatively high electrical impedance, it minimizes current flow into the electrically conductive fluid). This ionization, under optimal conditions, induces the discharge of energetic electrons and photons from the vapor layer and to the surface of the target tissue. A more detailed description of this phenomenon can be found in commonly assigned U.S. Pat. No. 5,683,366 the complete disclosure of which is incorporated herein by reference.

In the above procedure, it may also be desirable to effect collagen shrinkage, or contraction of the tissue layers underlying the removed or ablated epidermal tissue. In these procedures, the temperature of the electrode terminal(s) can be carefully controlled such that sufficient thermal energy is transferred to these underlying layers to contract the collagen connective tissue. The thermal energy may be transferred directly via RF current that passes through and resistively heats the underlying tissue layers, or it may be transferred indirectly by heating the electrically conductive fluid, and allowing the heated fluid to contact the underlying layers after the epidermal layers have been removed. A complete description of suitable methods of contracting collagen tissue with RF energy is described in U.S. patent application Ser. No. 08/942,580, filed on Oct. 2, 1997 (Attorney Docket No. 16238-001300), the complete disclosure of which is incorporated herein by reference.

In other procedures, it may be desired to treat vascular lesions, such as port wine stains, face veins, telangiectasis, birth marks, varicose veins and the like. In these procedures, electrical energy is applied to the vessel such that the energy is absorbed in the blood, which coagulates and collapses the vessel. The blood vessel may be accessed in a variety of manners. For example, high frequency voltage may be applied to one or more electrode terminals at the surface of the skin such that sufficient thermal energy is delivered through the skin to the blood vessel to coagulate the blood therein. Alternatively, the skin may be pierced with the sharpened tip of an electrosurgical probe. In this method, the probe is advanced to a location adjacent to the vessel to be treated, and high frequency electrical energy is applied to the distal end of the probe to coagulate and collapse the vessel at that location. This procedure may be repeated at multiple sites along the length of the vessel so that the vessel will collapse along its length.

In other methods, the high frequency voltage may be focused onto a small spot on the surface of the skin, over the vessel to be treated, such that a small volume of skin is ablated (e.g., to form a channel or hole) until the vessel is reached. Systems and methods for forming channels or holes through tissue with high frequency electrical energy are provided in commonly assigned U.S. Pat. No. 5,683,366.

In one method of the present invention, one or more electrode terminals are brought into close proximity to tissue at a target site, and the power supply is activated in the ablation mode such that sufficient voltage is applied between the electrode terminals and the return electrode to volumetrically remove the tissue through molecular dissociation, as described below. During this process, some vessels within the tissue may be severed. Smaller vessels will be automatically sealed with the system and method of the present invention. Larger vessels, and those with a higher flow rate, such as arterial vessels, may not be automatically sealed in the ablation mode. In these cases, the severed vessels may be sealed by activating a control (e.g., a foot pedal) to reduce the voltage of the power supply into the coagulation mode. In this mode, the electrode terminals may be pressed against the severed vessel to provide sealing and/or coagulation of the vessel. Alternatively, a coagulation electrode located on the same or a different instrument may be pressed against the severed vessel. Once the vessel is adequately sealed, the surgeon activates a control (e.g., another foot pedal) to increase the voltage of the power supply back into the ablation mode.

The present invention is also useful for cutting or incising tissue around nerves, such as cranial nerves, e.g., facial nerves, vestibulocochlear nerves, and the like. One of the significant drawbacks with prior art electrosurgical devices and lasers is that these devices do not differentiate between the target tissue and the surrounding nerves or bone. Therefore, the surgeon must be extremely careful during these procedures to avoid damage to the nerves within and around the target site. In the present invention, the Coblation™ process for removing tissue results in extremely small depths of collateral tissue damage as discussed above. This allows the surgeon to remove or cut tissue close to a nerve without causing collateral damage to the nerve fibers.

In addition to the generally precise nature of the novel mechanisms of the present invention, applicant has discovered an additional method of ensuring that adjacent nerves are not damaged during tissue removal. According to one aspect of the present invention, systems and methods are provided for distinguishing between the fatty tissue immediately surrounding nerve fibers and the “normal” target tissue that is to be removed during the procedure. Peripheral nerves usually comprise a connective tissue sheath, or epineurium, enclosing the bundles of nerve fibers, each bundle being surrounded by its own sheath of connective tissue (the perineurium) to protect these nerve fibers. The outer protective tissue sheath, or epineurium, typically comprises a fatty material having substantially different electrical properties than many target tissues. In one embodiment, an electrosurgical system of the present invention measures the electrical properties of the tissue at the tip of the probe with one or more sensing electrodes. These electrical properties may include electrical conductivity at one, several, or a range of frequencies (e.g., in the range from 1 kHz to 100 MHz); dielectric constant; capacitance; or combinations of these. In this embodiment, an audible signal may be produced when the sensing electrode(s) detect(s) the fatty tissue surrounding a nerve. Alternatively, direct feedback control can be provided to supply power to the electrode terminal(s), either individually or to the complete array of electrodes, only if and when the tissue encountered at the tip or working end of the probe is normal tissue based on the measured electrical properties.

In addition to the above, applicant has discovered that the Coblation™ mechanism of the present invention can be manipulated to ablate or cut certain tissue structures, while having little effect on other tissue structures. As discussed above, the present invention uses a technique of vaporizing electrically conductive fluid to form a plasma layer or pocket around the electrode terminal(s), and then inducing the discharge of energy from this plasma or vapor layer to break the molecular bonds of the target tissue components. Based on initial experiments, applicants believe that the free electrons within the ionized vapor layer are accelerated in the high electric fields near the electrode tip(s). When the density of the vapor layer (or within a bubble formed in the electrically conductive liquid) becomes sufficiently low (i.e., less than approximately 10²⁰ atoms/cm³ for aqueous solutions), the electron mean free path increases to enable subsequently injected electrons to cause impact ionization within these regions of low density (i.e., vapor layers or bubbles). Energy evolved by the energetic electrons (e.g., 4 to 5 eV) can subsequently bombard a molecule and break its bonds, dissociating a molecule into free radicals, which then combine into final gaseous or liquid species.

The energy evolved by the energetic electrons may be varied by adjusting a variety of factors, such as: the number of electrode terminals; electrode size and spacing; electrode surface area; asperities and sharp edges on the electrode surfaces; electrode materials; applied voltage and power; current limiting means, such as inductors; electrical conductivity of the fluid in contact with the electrodes; density of the fluid; and other factors. Accordingly, these factors can be manipulated to control the energy level of the excited electrons. Since different tissue structures have different molecular bonds, the present invention can be configured to break the molecular bonds of certain tissue, while having too low an energy to break the molecular bonds of other tissue. For example, fatty tissue, (e.g., adipose) tissue has double bonds that require a substantially higher energy level than 4 to 5 eV to break. Accordingly, the present invention in its current configuration generally does not ablate or remove such fatty tissue. However, the present invention may be used to effectively ablate cells to release the inner fat content in a liquid form. Of course, factors may be changed such that these double bonds can also be broken in a similar fashion as the single bonds (e.g., increasing voltage or changing the electrode configuration to increase the current density at the electrode tips). A more complete description of this phenomena can be found in co-pending U.S. patent application Ser. No. 09/032,375, filed Feb. 27, 1998 (Attorney Docket No. CB-3), the complete disclosure of which is incorporated herein by reference.

The present invention also provides systems, apparatus, and methods for selectively removing tumors, e.g., facial tumors, or other undesirable body structures while minimizing the spread of viable cells from the tumor. Conventional techniques for removing such tumors generally result in the production of smoke in the surgical setting, termed an electrosurgical or laser plume, which can spread intact, viable bacterial cells, viral particles, or neoplastic cells from the tumor or lesion to the surgical team, or to other portions of the patient's body. This potential spread of viable cells or particles has resulted in increased concerns over the proliferation of certain debilitating and fatal diseases, such as hepatitis, herpes, HIV and papillomavirus. In the present invention, high frequency voltage is applied between the electrode terminal(s) and one or more return electrode(s) to volumetrically remove at least a portion of the tissue cells in the tumor through the dissociation or disintegration of organic molecules into non-viable atoms and/or low molecular weight chemical species. Specifically, the present invention converts the solid tissue cells into non-condensable gases that are no longer intact or viable, thus avoiding the spread of viable tumor cells or infectious agents to other portions of the patient's body or to the surgical staff. The high frequency voltage is preferably selected to effect controlled removal of target tissue, while minimizing or completely avoiding necrosis or damage to surrounding or underlying non-target tissue. A more complete description of this phenomenon can be found in co-pending U.S. patent application Ser. No. 09/109,219, filed Jun. 30, 1998 (Attorney Docket No. CB-1), the complete disclosure of which is incorporated herein by reference

In one embodiment of the invention, the electrosurgical probe comprises a shaft or a hand-piece having a proximal end and a distal end, and one or more electrode terminal(s). The shaft or hand-piece may assume a wide variety of configurations, with the primary purpose being to mechanically support the active electrode terminals and permit the treating physician to manipulate the electrode from a proximal end of the shaft. For certain dermatology procedures, the shaft will typically have a length and diameter that facilitate handling by the surgeon.

The present invention may use a single active electrode terminal or an electrode array. In the latter situation, the array may be distributed over a contact surface of a probe. In one embodiment, an electrode array may include a plurality of independently current-limited and/or power-controlled electrode terminals for selectively applying apply electrical energy to the target tissue, while limiting the unwanted application of electrical energy to the surrounding tissue and environment resulting from power dissipation into surrounding electrically conductive liquids, such as blood, normal saline, electrically conductive gel, and the like. The electrode terminals may be independently current-limited by isolating the terminals from each other and connecting each terminal to a separate power source that is isolated from the other electrode terminals. Alternatively, the electrode terminals may be connected to each other at either the proximal or distal ends of the probe to form a single wire that couples to a power source.

The electrode terminal(s) are preferably supported within or by an insulating support positioned near the distal end of the instrument shaft. The return electrode may be located on the instrument shaft, on another instrument, or on the external surface of the patient (i.e., a dispersive pad). The close proximity of nerves and other sensitive tissue on the face and scalp makes a bipolar design more preferable because this minimizes the current flow through facial tissue and surrounding nerves. Accordingly, the return electrode is preferably either integrated with the instrument, or on a separate instrument to be positioned in close proximity to the distal end of the instrument. The proximal end of the instrument will include the appropriate electrical connections for coupling the return electrode(s) and the electrode terminal(s) to a high frequency power supply, such as an electrosurgical generator.

The current flow path between the electrode terminals and the return electrode(s) may be generated by submerging the tissue site in an electrical conducting fluid (e.g., within a viscous fluid, such as an electrically conductive gel), or by directing an electrically conductive fluid along a fluid path to the target site (i.e., a liquid, such as isotonic saline, or a gas, such as argon). The conductive gel may also be delivered to the target site to achieve a slower more controlled delivery rate of conductive fluid. In addition, the viscous nature of the gel may allow the surgeon to more easily contain the gel around the target site (e.g., rather than attempting to contain isotonic saline). A more complete description of an exemplary method of directing electrically conductive fluid between the active and return electrodes is described in U.S. Pat. No. 5,697,281, previously incorporated herein by reference. Alternatively, the body's natural conductive fluids, such as blood, may be sufficient to establish a conductive path between the return electrode(s) and the electrode terminal(s), and to provide the conditions for establishing a vapor layer, as described above. However, conductive fluid that is introduced to the patient is generally preferred over blood because blood will tend to coagulate at certain temperatures. Advantageously, a liquid electrically conductive fluid (e.g., isotonic saline) may be used to concurrently “bathe” the target tissue surface to provide an additional means for removing any tissue, and to cool the region of the tissue surrounding the site of ablation.

The power supply may include a fluid interlock for interrupting power to the electrode terminal(s) when there is insufficient conductive fluid around the electrode terminal(s). This ensures that the instrument will not be activated when conductive fluid is not present, minimizing the tissue damage that may otherwise occur. A more complete description of such a fluid interlock can be found in commonly assigned, co-pending U.S. application Ser. No. 09/058,336, filed Apr. 10, 1998 (attorney Docket No. CB-4), the complete disclosure of which is incorporated herein by reference.

In some procedures, it may also be necessary to retrieve or remove, e.g., via aspiration, the electrically conductive fluid, the non-condensable gaseous products of ablation, and/or tissue fragments that have not been completed ablated in situ. For example, in blepharoplasty procedures, it may be desired to remove the underlying fatty tissue from the patient's eyelids with the present invention. This may be accomplished by first breaking down this tissue with the Coblation® mechanism of the present invention, and then aspirated the remaining tissue fragments from the patient. Accordingly, the system of the present invention may include one or more suction lumen(s) in the instrument, or on a separate instrument, coupled to a suitable vacuum source for aspirating fluids from the target site. In addition, the invention may include one or more aspiration electrode(s), e.g., located within or adjacent to the distal end of the suction lumen, for ablating, or at least reducing the volume of, tissue fragments that are aspirated into the lumen. The aspiration electrode(s) function mainly to inhibit clogging of the lumen that may otherwise occur as larger tissue fragments are drawn therein. The aspiration electrode(s) may be different from the ablation electrode terminal(s), or the same electrode(s) may serve both functions. A more complete description of instruments incorporating aspiration electrode(s) can be found in U.S. Pat. No. 6,190,381, the complete disclosure of which is incorporated herein by reference.

In one configuration, each individual electrode terminal in the electrode array is electrically insulated from all other electrode terminals in the array of the probe, and is connected to a power source which is isolated from each of the other electrode terminals in the array, or to circuitry which limits or interrupts current flow to the electrode terminal when low resistivity material (e.g., blood, electrically conductive saline irrigant, or electrically conductive gel) causes a lower impedance path between the return electrode and the individual electrode terminal. The isolated power sources for each individual electrode terminal may be separate power supply circuits having internal impedance characteristics which limit power to the associated electrode terminal when a low impedance return path is encountered. By way of example, the isolated power source may be a user selectable constant current source. In this embodiment, lower impedance paths will automatically result in lower resistive heating levels since the heating is proportional to the square of the operating current times the impedance. Alternatively, a single power source may be connected to each of the electrode terminals through independently actuatable switches, or by independent current limiting elements, such as inductors, capacitors, resistors and/or combinations thereof. The current limiting elements may be provided in the probe, connectors, cable, controller or along the conductive path from the controller to the distal tip of the probe. Alternatively, the resistance and/or capacitance may occur on the surface of the active electrode terminal(s) due to oxide layers which form selected electrode terminals (e.g., titanium or a resistive coating on the surface of metal, such as platinum).

The tip region of the probe may comprise many independent electrode terminals designed to deliver electrical energy in the vicinity of the tip. The selective application of electrical energy to the conductive fluid is achieved by connecting each individual electrode terminal and the return electrode to a power source having independently controlled or current limited channels. The return electrode may be a tubular member of conductive material proximal to the electrode array at the tip which also serves as a conduit for the supply of the electrically conductive fluid between the active and return electrodes. The application of high frequency voltage between the return electrode and the electrode array results in the generation of high electric field intensities at the distal tips of the electrode terminals, with conduction of high frequency current from each individual electrode terminal to the return electrode. The current flow from each individual electrode terminal to the return electrode is controlled by either active or passive means, or a combination thereof, to deliver electrical energy to the electrically conductive fluid while minimizing energy delivery to surrounding (non-target) tissue.

In one embodiment, the application of a high frequency voltage between the return electrode and the electrode array for appropriate time intervals effects heating of the conductive fluid and contraction of the target tissue. The tissue volume over which energy is dissipated (i.e., a high current density exists) may be precisely controlled, for example, by the use of a multiplicity of small electrode terminals whose effective diameters or principal dimensions range from about 0.01 mm to 10 mm, preferably from about 5 mm to 0.05 mm, and more preferably from about 3 mm to 0.1 mm. Electrode areas for both circular and non-circular terminals typically have a contact area (per electrode terminal) below 25 mm₂, preferably being in the range from 0.0001 mm² to 1 mm², and more preferably from 0.005 mm² to 0.5 mm². The circumscribed area of the electrode array is in the range from 0.25 mm² to 75 mm², preferably from 0.5 mm² to 40 mm², and will usually include at least two isolated electrode terminals and sometimes up to about 50 or more electrode terminals, disposed on the distal surfaces of the instrument. The use of small diameter electrode terminals increases the electric field intensity and reduces the extent, or depth, of tissue heating as a consequence of the divergence of current flux lines which emanate from the exposed surface of each electrode terminal.

The electrode terminal(s) are typically disposed on a tissue treatment surface on the shaft of the electrosurgical probe or instrument. In one embodiment, the return electrode surface may be recessed relative to the distal end of the probe and may be recessed within a fluid conduit provided for the introduction of electrically conductive fluid to the site of the target tissue and electrode terminal(s).

The area of the tissue treatment surface can vary widely, and the tissue treatment surface can assume a variety of geometries, with particular areas and geometries being selected for specific applications. Active electrode surfaces typically have areas in the range from 0.25 mm² to 75 mm², usually being from about 0.5 mm² to 40 mm². The geometries can be planar, concave, convex, hemispherical, conical, linear “in-line” array, or virtually any other regular or irregular shape. Most commonly, the active electrode(s) or electrode terminal(s) are disposed at the distal end of the electrosurgical probe shaft, frequently being planar, disk-shaped, or hemispherical surfaces for use in reshaping procedures, or being linear arrays, conical, or cylindrical for use in cutting, excising, or forming voids in target tissue. The distal end or treatment surface of the instrument may be beveled. Alternatively or additionally, the active electrode(s) may be formed on lateral surfaces of the electrosurgical probe shaft (e.g., in the manner of a spatula), facilitating access to certain body structures in endoscopic procedures.

In one embodiment, the electrode array comprises a plurality of substantially elongate electrode terminals spaced on a contact surface of a shaft. The contact surface may comprise an electrically insulating electrode support member extending from the shaft of the probe. The elongate electrode terminals will typically have a length of about 0.5 to 30 mm, usually about 1 to 15 mm, and often about 3 to 7 mm. The width of the elongate electrode terminals is usually about 0.01 to 2 mm, typically about 0.05 to 1 mm, and often about 0.1 to 0.5 mm. Typically the elongate electrode terminals are spaced from each other by a distance of about 0.05 to 4 mm, preferably about 0.1 mm to 4 mm. The electrode array may comprise from one electrode terminal to more than 50 electrode terminals. Electrode terminals of the invention provide a substantially uniform application of energy to the tissue at the treatment site.

In one embodiment, the electrode support comprises a plurality of wafer layers bonded together, e.g., by a glass adhesive or the like. The wafer layers each have conductive strips printed thereon to form the electrode terminal(s) and the return electrode(s). In one embodiment, the proximal end of the wafer layers will have a number of holes extending from the conductor strips to an exposed surface of the wafer layers for connection to electrical conductor lead traces in the electrosurgical probe or hand-piece. The wafer layers preferably comprise a ceramic material , such as alumina, and the electrode will preferably comprise a metallic material, such as gold, platinum, palladium, tungsten, silver or the like. Suitable multilayer ceramic electrodes are commercially available from e.g., VisPro Corporation of Beaverton, Oreg.

The electrically conductive fluid should have a threshold conductivity to provide a suitable conductive path between the return electrode and the electrode terminal(s). The electrical conductivity of the fluid (in units of milliSiemens per centimeter or mS/cm) is usually greater than about 0.2 mS/cm, preferably greater than 2 mS/cm, and more preferably greater than 10 mS/cm. In an exemplary embodiment, the electrically conductive fluid is isotonic saline, which has a conductivity of about 17 mS/cm. Alternatively, the fluid may be an electrically conductive gel or spray, such as a saline electrolyte gel, a conductive ECG spray, an electrode conductivity gel, an ultrasound transmission or scanning gel, or the like. Suitable gels or sprays are commercially available from Graham-Field, Inc. of Hauppauge, N.Y.

In some embodiments, the electrode support and the fluid outlet may be recessed from an outer surface of the probe or hand-piece to confine the electrically conductive fluid to the region immediately surrounding the electrode support. In addition, the shaft may be shaped so as to form a cavity around the electrode support and the fluid outlet. This helps to ensure that the electrically conductive fluid will remain in contact with the electrode terminal(s) and the return electrode(s) to maintain the conductive path therebetween. In this manner, a vapor or plasma layer is maintained between the electrode terminal(s) and the tissue at the treatment site throughout the procedure, which reduces the thermal damage that might otherwise occur if the vapor layer were extinguished due to a lack of conductive fluid. The electrically conductive fluid also helps maintain the tissue at a suitable temperature during the procedure.

The voltage applied between the return electrode and the electrode array is at high- or radio frequency, typically between about 5 kHz and 20 MHz, usually being between about 30 kHz and 2.5 MHz, preferably being between about 50 kHz and 500 kHz, more preferably less than 350 kHz, and most often between about 100 kHz and 200 kHz. The RMS (root mean square) voltage applied will usually be in the range from about 5 volts to 1000 volts, preferably being in the range from about 10 volts to 500 volts depending on the electrode terminal size, the operating frequency, and the operation mode of the particular procedure or desired effect on the tissue (i.e., contraction, coagulation, or ablation). Typically, the peak-to-peak voltage is in the range of 10 to 2000 volts and preferably in the range of 20 to 1200 volts, and more preferably in the range of about 40 to 800 volts (again, depending on the electrode size, the operating frequency, and the operation mode).

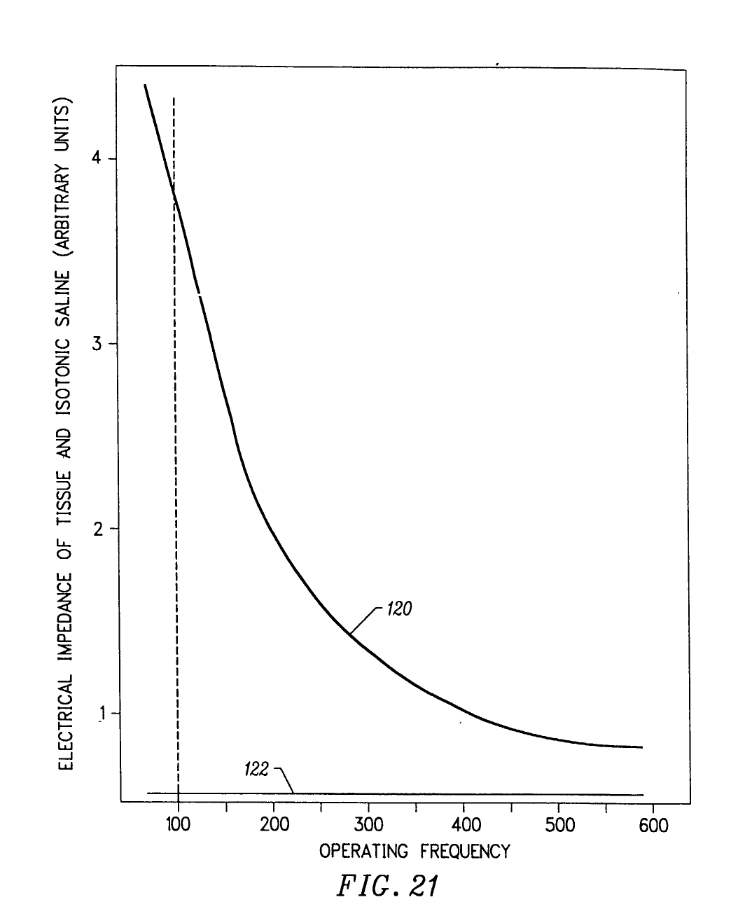

An important aspect of the present invention is the discovery that the frequency of the output voltage of the generator can be selected to control the depth of tissue heating. Referring to FIG. 21, the electrical impedance of tissue is known to decrease with increasing frequency due to the electrical properties of cell membranes which surround electrically conductive cellular fluid. As shown, the electrical impedance of tissue to current at a frequency of 100 kHz is approximately four times larger than at a frequency of 450 to 500 kHz. As a result of the higher tissue impedance, the current flux lines tend to penetrate less deeply resulting in a smaller depth of tissue heating. This principle of operation of the present invention can be used to advantage in applications where the depth of tissue heating is to be maintained small (e.g., 0.2 to 0.5 mm). Typically, the operating frequency is less than 350 kHz for applications requiring shallow depths of tissue heating (e.g., less than 1.5 mm). Conversely, in situations where much larger depths of tissue heating are to be effected, a higher output voltage frequency may be used. By way of example, to achieve therapeutic collagen shrinkage to a depth of 1.5 to 3.0 mm, a higher operating frequency may be used (e.g., 500 kHz). Alternatively, the diameter of the electrode terminals and/or the spacing between the outer perimeter of the electrode terminals and the electrode support member may be selected to increase the depth of current penetration. By way of example, increasing the distance between the outer perimeter of the support member and the electrode terminals will increase the depth of heating for a given operating frequency.

As discussed above, the voltage is usually delivered in a series of voltage pulses or alternating current of time varying voltage amplitude with a sufficiently high frequency (e.g., on the order of 5 kHz to 20 MHz) such that the voltage is effectively applied continuously (as compared with e.g., lasers claiming small depths of necrosis, which are generally pulsed about 10 to 20 Hz). In addition, the duty cycle (i.e., cumulative time in any one-second interval that energy is applied) is on the order of about 50% for the present invention, as compared with pulsed lasers which typically have a duty cycle of about 0.0001%.

The preferred power source of the present invention delivers a high frequency current selectable to generate average power levels ranging from several milliwatts to tens of watts per electrode, depending on the volume of target tissue being heated, the total number of electrode(s), and/or the maximum allowed temperature selected for the probe tip. The power source allows the user to select the voltage level according to the specific requirements of a particular arthroscopic surgery, cosmetic surgery, dermatological procedure, ophthalmic procedures, open surgery or other endoscopic surgery procedure. A more detailed description of a power suitable for use in conjunction with the instant invention source can be found in U.S. Provisional Patent Application No. 60/062,997, filed on Oct. 23, 1997 (Attorney Docket No. 16238-007400), the complete disclosure of which is incorporated herein by reference.

The power source may be current limited or otherwise controlled so that undesired heating of the target tissue or surrounding (non-target) tissue does not occur. In one embodiment of the present invention, current limiting inductors are placed in series with each independent electrode terminal, where the inductance of the inductor is in the range of 10 uH to 50,000 uH, depending on the electrical properties of the target tissue, the size of the electrode terminal(s), the desired tissue heating rate, and the operating frequency. Alternatively, capacitor-inductor (LC) circuit structures may be employed, as described previously in PCT application No. PCT/US94/05168, the complete disclosure of which is incorporated herein by reference. Additionally, current limiting resistors may be selected. Preferably, these resistors will have a large positive temperature coefficient of resistance so that, as the current level begins to rise for any individual electrode terminal in contact with a low resistance medium (e.g., saline irrigant or conductive gel), the resistance of the current limiting resistor increases significantly, thereby minimizing the power delivery from the electrode terminal into the low resistance medium (e.g., saline irrigant or conductive gel).

It should be clearly understood that the invention is not limited to electrically isolated electrode terminals, or even to a plurality of electrode terminals. For example, the array of active electrode terminals may be connected to a single lead that extends through the probe shaft to a power source of high frequency current. Alternatively, the probe may incorporate a single electrode that extends directly through the probe shaft or is connected to a single lead that extends to the power source.

During a surgical procedure, the distal end of the probe or the electrode terminal(s) may contact the target tissue, or may be maintained at a small distance away from the target tissue surface. This small spacing allows for the continual re-supply of electrically conductive fluid into the interface between the electrode terminal(s) and the target tissue surface. This continual re-supply of the electrically conductive fluid helps to ensure that the thin vapor layer will remain between electrode terminal(s) and the tissue surface. In addition, dynamic movement of the electrode terminal(s) over the tissue site allows the electrically conductive fluid to cool the tissue underlying and surrounding the target tissue to minimize thermal damage to this surrounding and underlying tissue. To that end, the electrically conductive fluid may be cooled to facilitate this cooling of the tissue. Typically, the active electrode(s) are about 0.02 to 2 mm from the target tissue, and more typically about 0.05 to 0.5 mm, during the ablation process. One method of maintaining this space is to translate and/or rotate the probe transversely relative to the tissue, i.e., a light brushing motion, to maintain a thin vaporized layer or region between the active electrode and the tissue. Of course, if coagulation or collagen shrinkage of a deeper region of tissue is necessary (e.g., for sealing a bleeding vessel imbedded within the tissue), it may be desirable to press the electrode terminal(s) against the tissue to effect joulean heating therein.

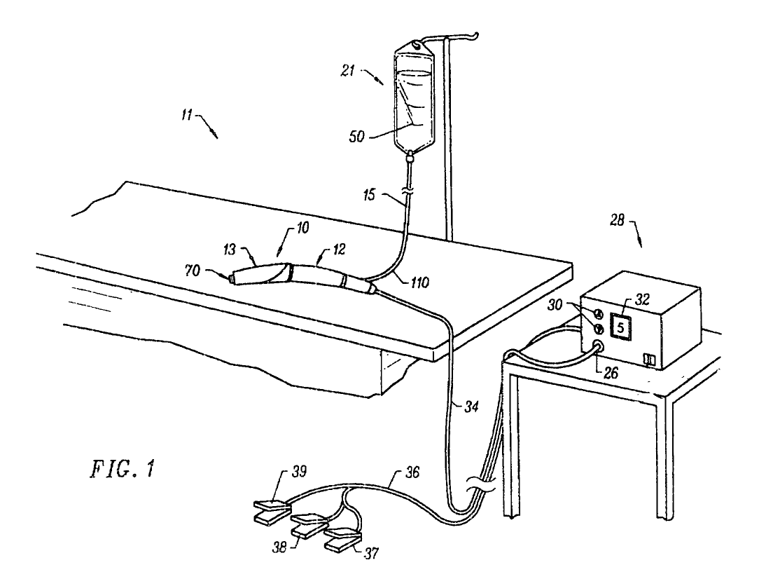

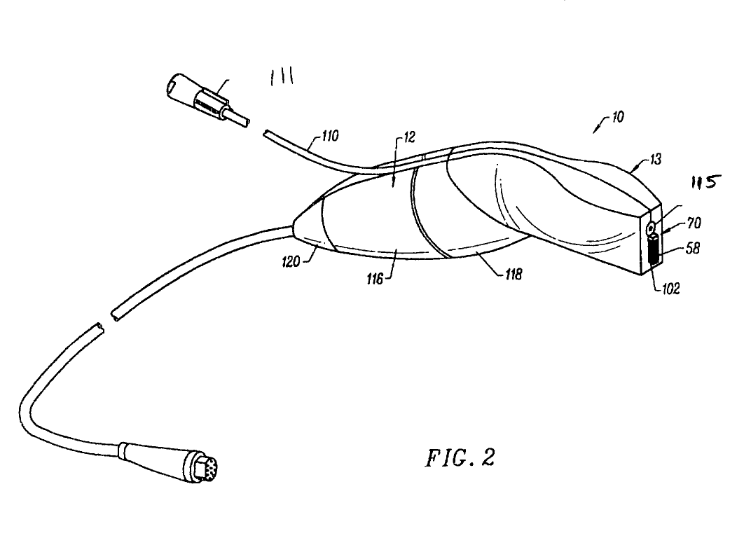

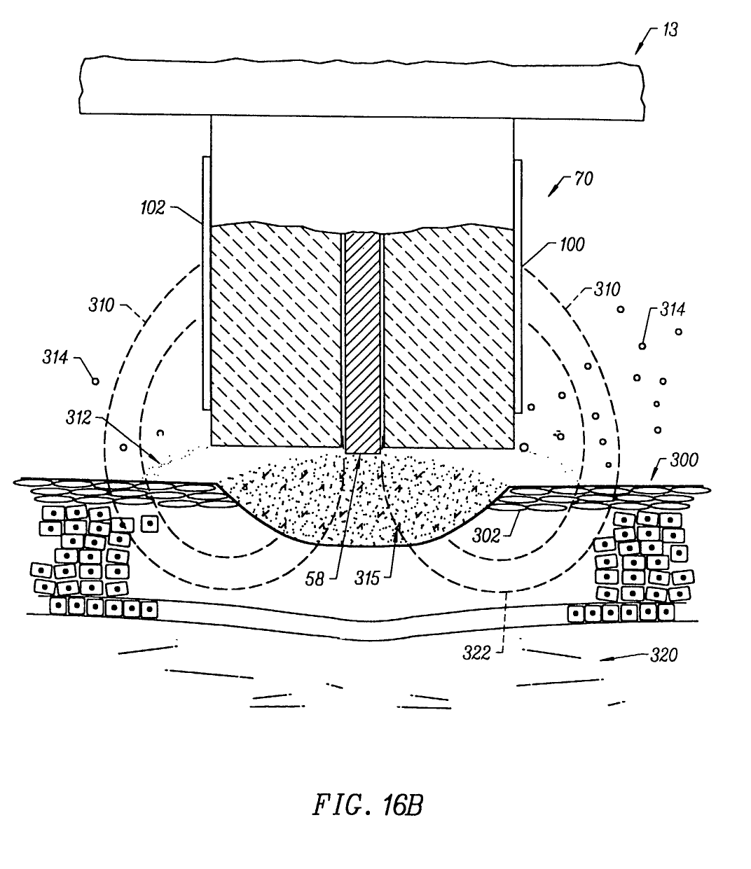

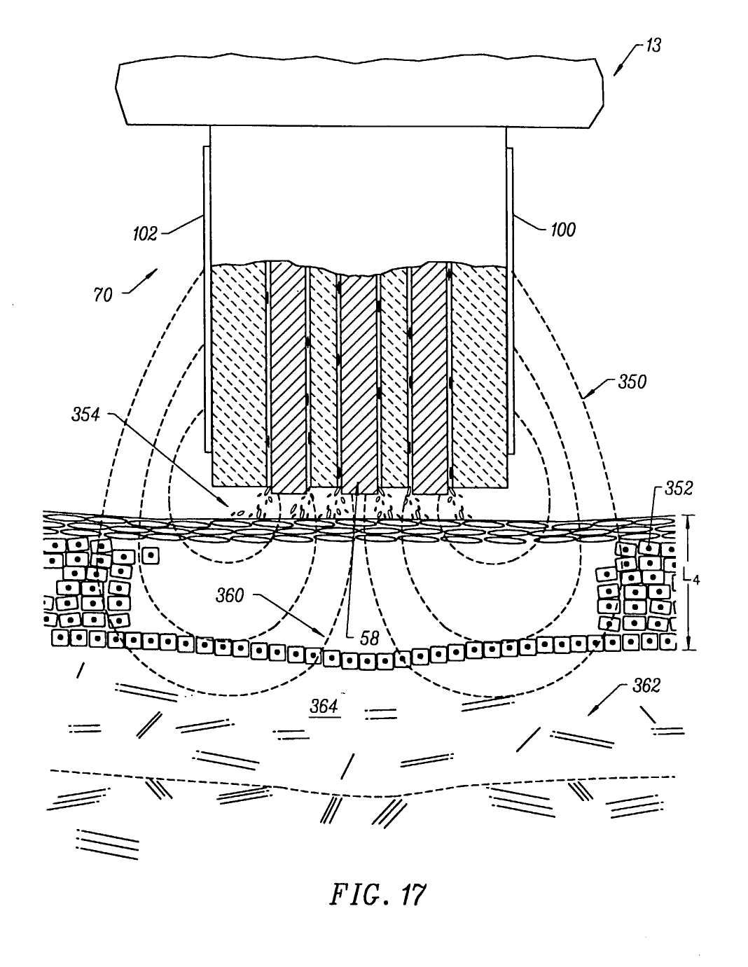

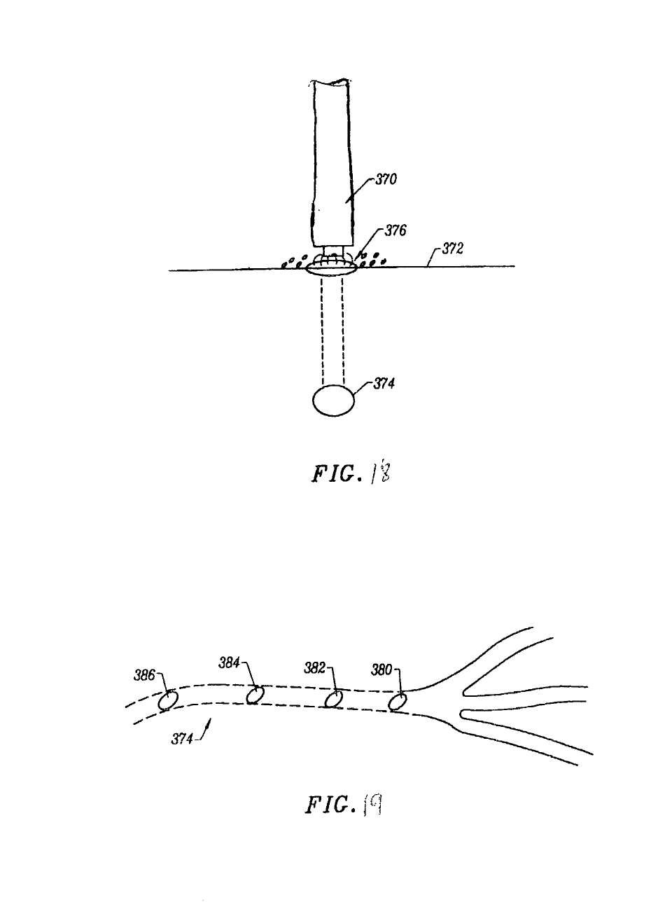

Referring to FIG. 1, an electrosurgical system 11 generally comprises an electrosurgical hand-piece or probe 10 connected to a power supply 28 for providing high frequency voltage to a target site, and a fluid source 21 for supplying electrically conductive fluid 50 to probe 10. Probe 10 generally includes a proximal handle 12 and a distal tip 13 having an electrode support member 70 with one electrode terminal 58, or an array of electrode terminals 58, and one or more return electrodes 100, 102 (see FIGS. 2, 4 and 5) disposed on the support member 70. A connecting cable 34 has a connector 26 for electrically coupling the electrodes in probe 10 to power supply 28. The electrode terminals 58 are electrically isolated from each other and each of the terminals 58 is connected to an active or passive control network within power supply 28 by means of a plurality of individually insulated conductors (not shown). A fluid supply tube 15 is connected to a fluid tube 110 of probe 10 for supplying electrically conductive fluid 50 to the distal tip 13 (see FIGS. 16 and 17).

Power supply 28 has an operator controllable voltage level adjustment 30 to change the applied voltage level, which is observable at a voltage level display 32. Power supply 28 also includes first, second, and third foot pedals 37, 38, 39 and a cable 36 which is removably coupled to power supply 28. The foot pedals 37, 38, 39 allow the surgeon to remotely adjust the energy level applied to electrode terminals 58. In an exemplary embodiment, first foot pedal 37 is used to place the power supply into the “ablation” mode and second foot pedal 38 places power supply 28 into the “coagulation” mode. The third foot pedal 39 allows the user to adjust the voltage level within the “ablation” mode. In the ablation mode, a sufficient voltage is applied to the electrode terminals 58 to establish the requisite conditions for molecular dissociation of the tissue (i.e., vaporizing a portion of the electrically conductive fluid, ionizing the vapor layer, and accelerating charged particles from the vapor layer against the tissue). As discussed above, the requisite voltage level for ablation will vary depending on the number, size, shape and spacing of the electrodes, the distance to which the electrodes extend from the support member, etc. When the surgeon is using the power supply in the “ablation” mode, voltage level adjustment 30 or third foot pedal 39 may be used to adjust the voltage level to adjust the degree or aggressiveness of the ablation.

Of course, it will be recognized that the voltage and modality of the power supply may be controlled by other input devices. However, applicant has found that foot pedals are convenient methods of controlling the power supply while manipulating the probe during a surgical procedure.

In the coagulation mode, the power supply 28 applies a low enough voltage to one or more electrode terminals (or one or more coagulation electrodes) to avoid vaporization of the electrically conductive fluid, formation of a plasma and subsequent molecular dissociation of the tissue. The surgeon may automatically toggle the power supply between the ablation and coagulation modes by alternatively stepping on foot pedals 37, 38, respectively. This allows the surgeon to quickly move between coagulation and ablation in situ, without having to remove his/her concentration from the surgical field or without having to request an assistant to switch the power supply. By way of example, as the surgeon is sculpting soft tissue in the ablation mode, the probe typically will simultaneously seal and/or coagulate small severed vessels within the tissue. However, larger vessels, or vessels with high fluid pressures (e.g., arterial vessels) may not be sealed in the ablation mode. Accordingly, the surgeon can simply step on foot pedal 38, automatically lowering the voltage level below the threshold level for ablation, and apply sufficient pressure onto the severed vessel for a sufficient period of time to seal and/or coagulate the vessel. After this is completed, the surgeon may quickly move back into the ablation mode by stepping on foot pedal 37. A specific design of a suitable power supply for use with the present invention can be found in U.S. Provisional Patent Application No. 60/062,997, filed Oct. 23, 1997 (attorney docket no. 16238-007400).

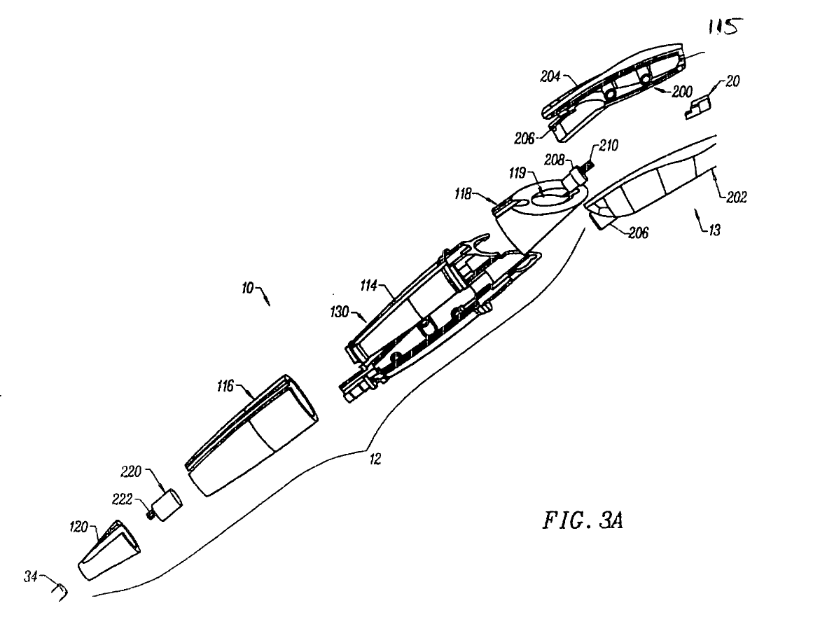

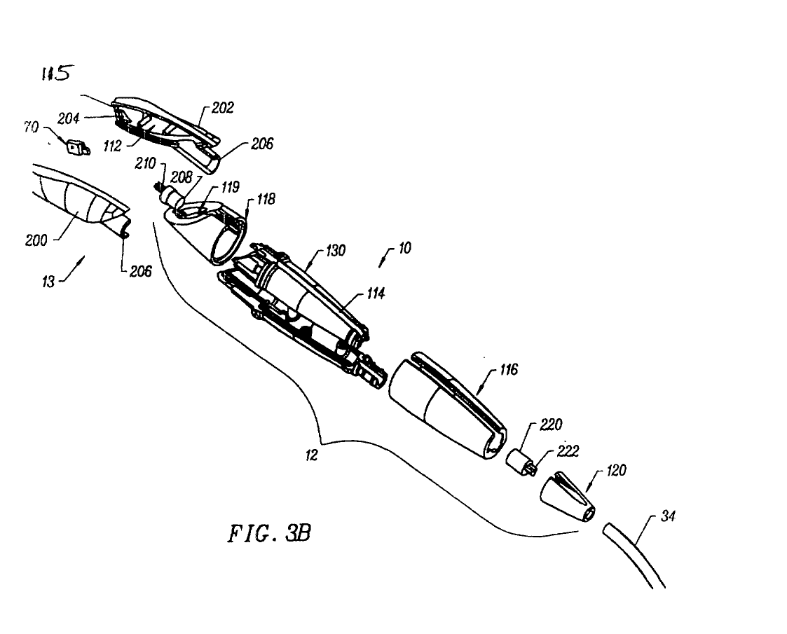

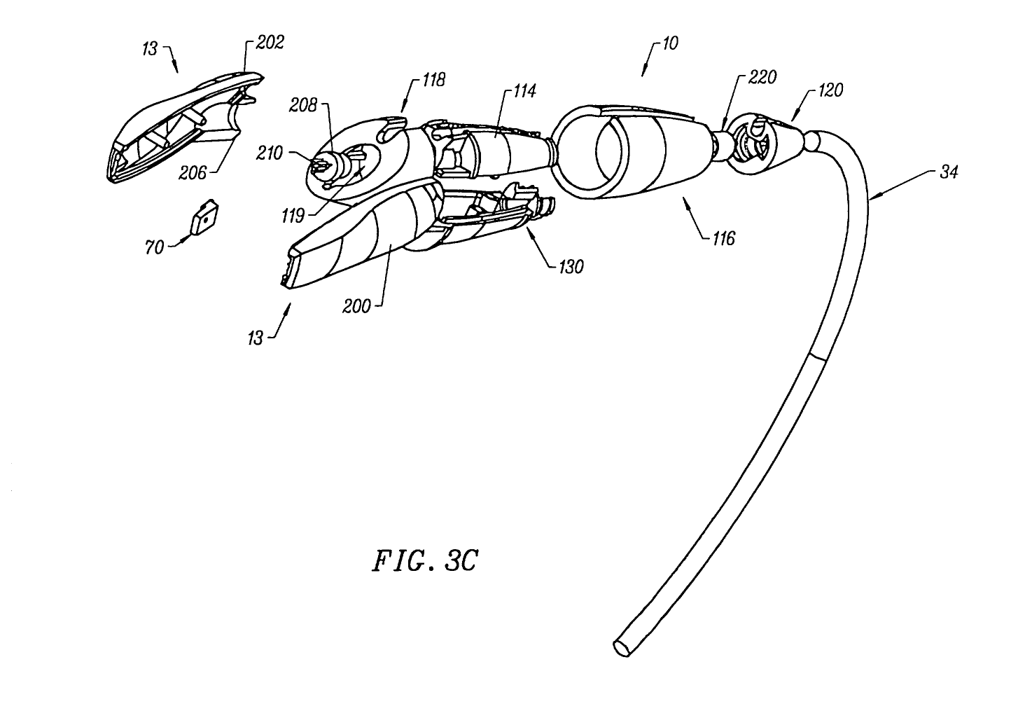

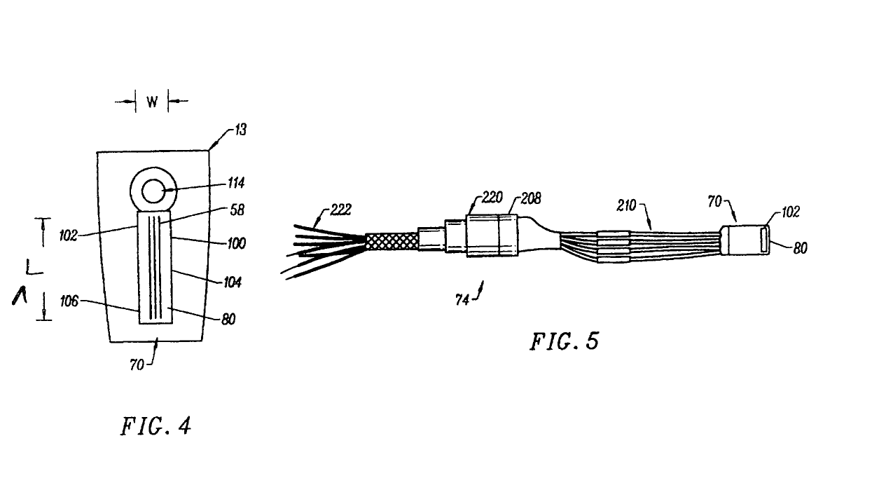

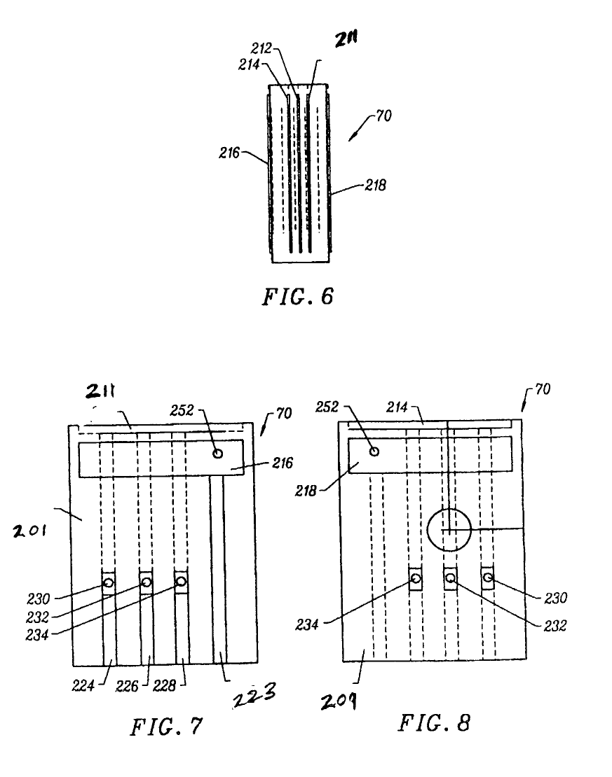

Referring now to FIGS. 2-5, an exemplary electrosurgical probe 10 comprises a shaft or disposable tip 13 removably coupled to a proximal handle 12, and an electrically insulating electrode support member 70 extending from tip 13 for supporting a plurality of electrode terminals 58 (see FIGS. 2 and 5). Tip 13 and handle 12 typically comprise a plastic material that is easily molded into a suitable shape for handling by the surgeon. As shown in FIGS. 3A-C and FIG. 5, handle 12 defines an inner cavity 72 that houses the electrical connections 74 (discussed below with reference to FIG. 5), and provides a suitable interface for connection to electrical connecting cable 34 (see FIG. 1). In one embodiment, handle 12 is constructed of a steam autoclavable plastic or metal (e.g., polyethylether ketone, or a stable metal alloy containing aluminum and/or zinc) so that it can be re-used by sterilizing handle 12 between surgical procedures. High service temperature materials are preferred, such as a silicone cable jacket and a polyetherimide or ULTEM® hand-piece that can withstand repeated exposure to high temperatures.

Referring to FIGS. 3A-3C, tip 13 preferably comprises first and second housing halves 200, 202 that snap fit together, and form a recess 204 therebetween for holding electrode support member 70 within the tip 13. Electrode support member 70 extends from the distal end of tip 13 (usually by about 0.5 to 20 mm), and provides support for a plurality of electrically isolated electrode terminals 58 and one or more return electrodes 100, 102 (see FIG. 4). Alternatively, electrode support member 70 may be recessed from the distal end of tip 13 to help confine the electrically conductive fluid around the electrode terminals 58 during the surgical procedure, as discussed above. Electrode support member 70 has a substantially planar tissue treatment surface 80 that is usually disposed at an angle of about 10 to 90 degrees relative to the longitudinal axis of handle 12 to facilitate handling by the surgeon. In the exemplary embodiment, this function is accomplished by orienting tip 13 at an acute angle relative to the longitudinal axis of handle 12.

In the embodiment shown in FIGS. 2-5, probe 10 includes first and second return electrodes 100, 102 for completing the current path between electrode terminals 58 and power supply 28 (see FIG. 1). As shown, return electrodes 100, 102 preferably have fluid contact surfaces on either lateral surface 104, 106 of electrode support member 70 slightly proximal to tissue treatment surface 80, typically about 0.1 to 2 mm, preferably about 0.2 to 1 mm. Return electrodes 100, 102 will usually have an exposed surface area of about 5 mm² to 25 mm², preferably about 18 mm² to about 20 mm². Return electrodes 100, 102 are coupled to a connector 105 (details of this connection are discussed below) that extends to the proximal end of handle 13, where it is suitably connected to power supply 28 (FIG. 1).

Referring to FIGS. 3A-3C and FIG. 5, tip 13 further includes a proximal hub 206 for supporting a male electrical connector 208 that holds a plurality of wires 210 each coupled to one of the electrode terminals 58 and the return electrodes 100, 102 on support member 70 (see FIGS. 7-13 for details of the representative support member 70). A female connector 220 housed within handle 12 is removably coupled to male connector 208, and a plurality of wires 222 extend from female connector 220 through a strain relief 120 to cable 34. Both sets of wires 210, 222 are insulated to prevent shorting in the event of fluid ingress into the probe 10. This design allows for removable connection of the electrodes in tip 13 with the connector 220 within handle 12 so that the handle can be re-used with different tips 13. Probe 10 will preferably also include an identification element, such as a coded resistor (not shown), for programming a particular voltage output range and mode of operation for the power supply. This allows the power supply to be employed with a variety of different probes for a variety of different applications.

As shown in FIG. 5, return electrodes 100, 102 are not directly connected to electrode terminals 58. To complete this current path so that electrode terminals 58 are electrically connected to return electrodes 102, 100, electrically conductive fluid (e.g., isotonic saline or electrically conductive gel) is located between the active and return electrodes during a surgical procedure. In the representative embodiment, probe 10 includes a fluid tube 110 (FIG. 2) for delivering electrically conductive fluid to the target site. Fluid tube 110 is sized to extend through a groove 114 in handle 12 and through an inner cavity 112 (FIG. 2 and FIGS. 3A-3C) in tip 13 to a distal opening 115 (FIG. 4) located adjacent electrode support member 70. Tube 110 extends all the way through inner cavity 112 to opening 115 to eliminate any possible fluid ingress into cavity 112. As shown in FIGS. 1 and 2, fluid tube 110 includes a proximal connector 111 for coupling tube 110 to a source 21 of an electrically conductive fluid 50.

Probe 10 will also include a valve or equivalent structure for controlling the flow rate of the electrically conductive fluid to the target site. In the representative embodiment shown in FIGS. 3A-3C, handle 12 comprises a main body 130 coupled between distal hub 118 and strain relief 120, and a rotatable sleeve 116 around main body 130. Distal hub 118 has an opening 119 for receiving proximal hub 206 of tip 13 for removably coupling the tip 13 to the handle 12. Sleeve 116 is rotatably coupled to strain relief 120 and distal hub 118 to provide a valve structure for fluid tube 110. Fluid tube 110 extends through groove 114 from strain relief 120, through main body 130 and distal hub 118 to tip 13. Rotation of sleeve 116 will impede, and eventually obstruct, the flow of fluid through tube 110. Of course, this fluid control may be provided by a variety of other input and valve devices, such as switches, buttons, etc.

In alternative embodiments, the fluid path may be directly formed in probe 10 by, for example, an inner lumen or an annular gap (not shown) within the handle and the tip. This inner lumen may be formed near the perimeter of the probe 10 such that the electrically conductive fluid tends to flow radially inward towards the target site, or the inner lumen may be formed towards the center of probe 10 so that the fluid flows radially outward. In addition, the electrically conductive fluid may be delivered from a fluid delivery element (not shown) that is separate from probe 10. In arthroscopic surgery, for example, the joint cavity may be flooded with isotonic saline, and the probe 10 introduced into this flooded cavity. Electrically conductive fluid is continually re-supplied to maintain the conduction path between return electrodes 100, 102 and electrode terminals 58. A more complete description of alternative electrosurgical probes incorporating one or more fluid lumen(s) can be found in U.S. Pat. No. 5,697,281, the complete disclosure of which is incorporated herein by reference.

Referring to FIGS. 4 and 5, electrically isolated electrode terminals 58 are spaced apart over tissue treatment surface 80 of electrode support member 70. In the representative embodiment, the tissue treatment surface 80 has a rectangular cross-sectional shape with a length L in the range of about 0.5 mm to 20 mm (typically about 2 to 10 mm), and a width W in the range from 0.3 mm to 10 mm (typically about 0.5 to 4 mm). The individual electrode terminals 58 have the dimensions described above, and in one embodiment are substantially flush with tissue treatment surface 80. Applicant has found that this configuration minimizes any sharp electrode edges and/or corners that would promote excessively high electric field intensities and associated current densities when a high frequency voltage is applied to the electrode terminals, thereby preventing excessively high rates of ablation, as preferred for removing thin layers of tissue (e.g., epidermal layers).

It should be noted that the electrode terminals 58 may protrude slightly outward from surface 80, typically by a distance up to about 2 mm. Alternatively, electrode terminals 58 may be recessed from surface 80. For example, electrode terminals 58 may be recessed by a distance in the range of from about 0.01 mm to 1 mm, typically from about 0.1 mm to 0.2 mm. In one embodiment of the invention, the electrode terminals are axially adjustable relative to the tissue treatment surface so that the surgeon can adjust the distance between the treatment surface and the distal end of the electrode terminals.

Referring now to FIGS. 7-13, an exemplary electrode support member 70 will be described in detail. As shown, electrode support member 70 preferably comprises a multilayer substrate comprising a suitable high temperature, electrically insulating material, such as ceramic. The multilayer substrate is a thin- or thick-film hybrid having conductive strips that are adhered to the ceramic wafer layers (e.g., thick-film printed and fired onto or plated onto the ceramic wafers). The conductive strips typically comprise tungsten, gold, nickel, silver, platinum or equivalent materials. In the exemplary embodiment, the conductive strips comprise gold, and they are co-fired together with the wafer layers to form an integral package. The conductive strips are coupled to external wire connectors by holes or vias through the ceramic layers, and plated or otherwise covered with conductive material.

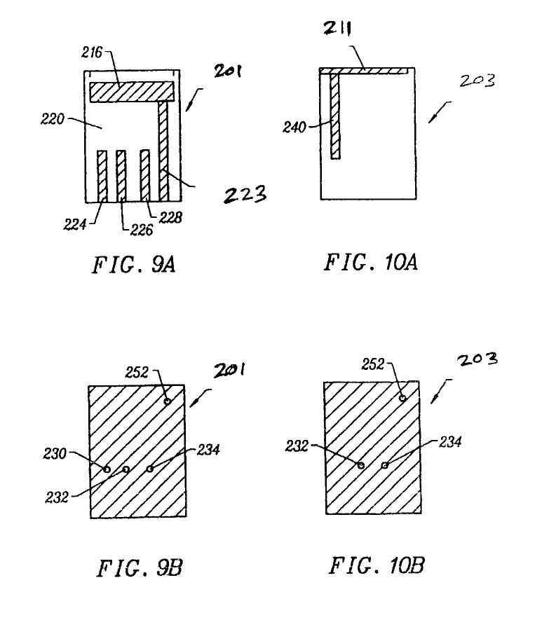

In the representative embodiment, support member 70 comprises five ceramic layers 201, 203, 205, 207, 209 (see FIGS. 9A-13), three gold plated electrode terminals 211, 212, 214 and first and second gold plated return electrodes 216, 218. As shown in FIGS. 8, 9A and 9B, a first ceramic layer 201, which is one of the outer layers of support 70, includes first gold plated return electrode 216 on a lateral surface 221 thereof. First ceramic layer 201 further includes a gold conductive strip 223 extending from return electrode 216 to the proximal end of the layer 201 for coupling to a lead wire (not shown), and three gold conductive lines 224, 226, 228 extending from a mid-portion of the layer 201 to its proximal end. Conductive strips 224, 226, 228 are each coupled to one of the electrode terminals 211, 212, 214 by conductive holes or vias 230, 232, 234, respectively. As shown, all three vias 230, 232, 234 extend through wafer layer 201.

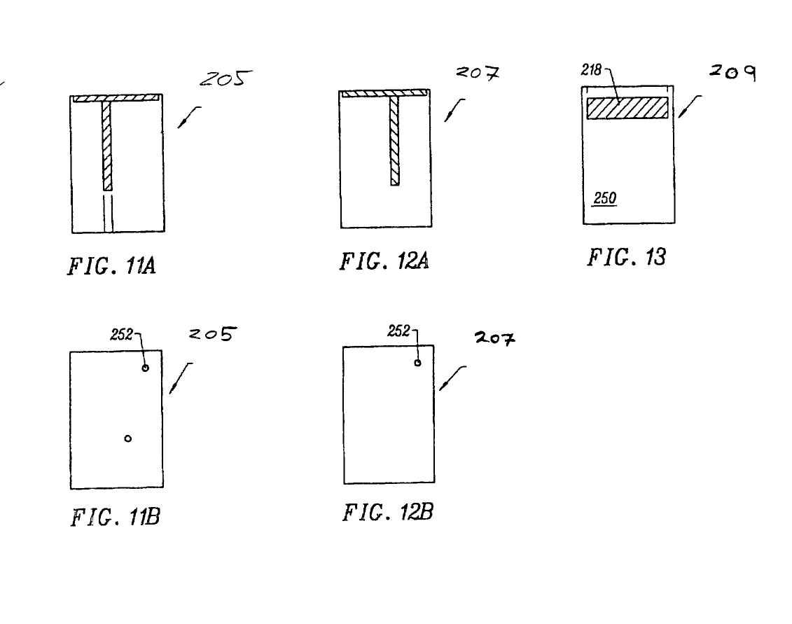

Referring to FIGS. 10A and 10B, a second wafer layer 203 is bonded between the outer wafer layer 201 and a middle wafer layer 205 (FIGS. 11A and 11B). As shown, first electrode terminal 211 is attached to the distal surface of second wafer layer 203, and a conductive strip 240 extends to via 230 to couple electrode terminal 211 to a lead wire. Similarly, wafer layers 205 and 207 (FIGS. 11A-12B) each have an electrode terminal 212, 214, respectively, plated to their distal surfaces; and a conductive strip 242, 244, respectively, extending to one of the vias 232, 234, respectively. Note that the vias only extend as far as necessary through the ceramic layers. As shown in FIG. 13, another outer wafer layer 209 has a second return electrode 218 plated to the lateral surface 250 of layer 209. The second return electrode 218 is coupled directly to the first return electrode 216 through a via 252 extending through the entire ceramic substrate.

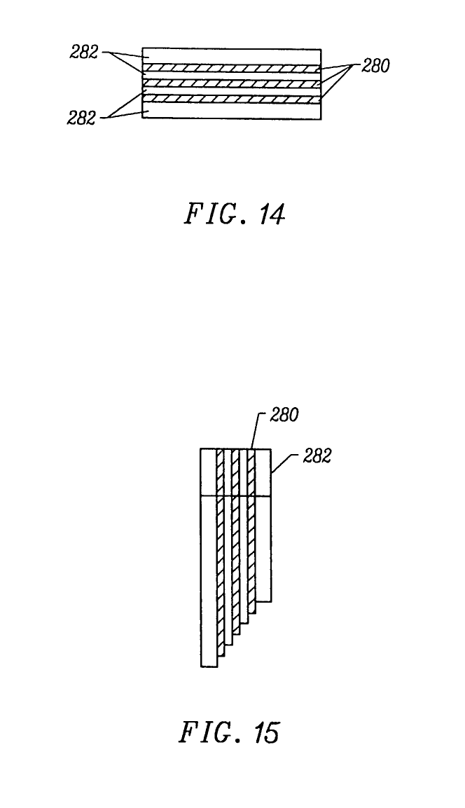

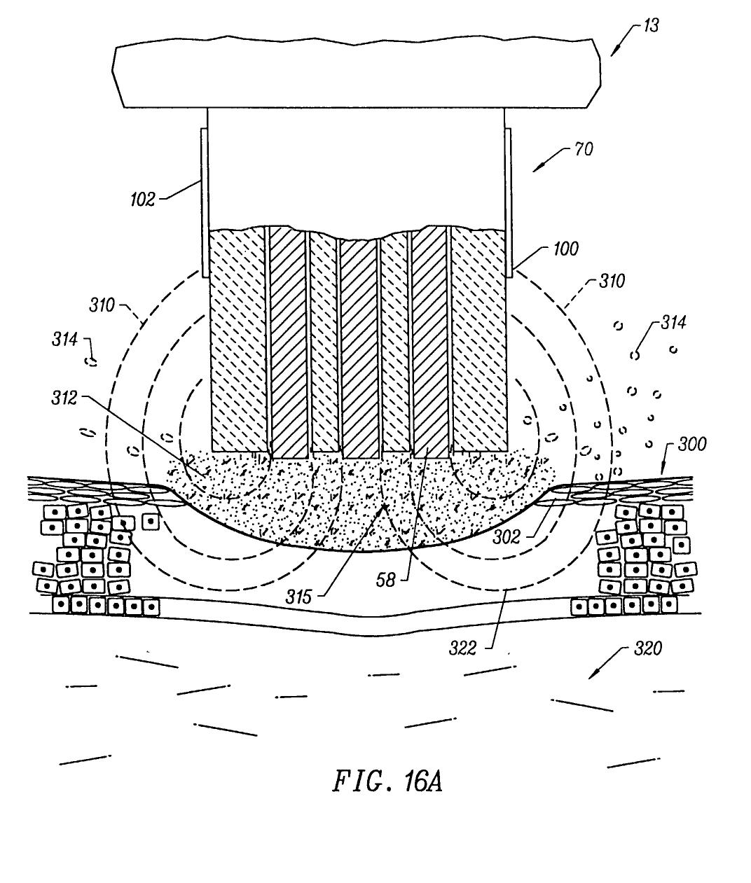

Of course, it will be recognized that a variety of different types of multilayer wafers may be constructed according to the present invention, For example, FIGS. 14 and 15 illustrate an alternative embodiment of the multilayer ceramic wafer, wherein the electrode terminals comprise planar strips 280 that are plated or otherwise bonded between the ceramic wafer layers 282. Each of the planar strips 280 has a different length, as shown in FIG. 15, so that the electrode terminals can be electrically isolated from each other, and coupled to lead wires by vias (not shown).

Referring now to FIGS. 16A and 16B, a method of treating tissue on the outer skin of a patient according to the present invention will now be described. As shown, distal tip 13 of probe 10 is positioned such that electrode support 70 is adjacent to the target tissue 302 at the treatment site 300. Electrically conductive fluid (e.g., FIG. 17) is delivered through fluid tube 110 (FIG. 2) through distal opening 115 to the treatment site 300. The rate of fluid flow is controlled with rotatable sleeve 116 (FIG. 3A) such that the zone between the tissue 302 and electrode support 70 is constantly immersed in the fluid. The power supply 28 is then turned on and adjusted such that a high frequency voltage difference is applied between electrode terminal(s) 58 and return electrodes 100, 102. The electrically conductive fluid provides the conduction path (see current flux lines 310) between electrode terminal(s) 58 and the return electrodes 100, 102 on either side of electrode support 70.

In the exemplary embodiment, the high frequency voltage is sufficient to convert the electrically conductive fluid between the target tissue 302 and electrode terminals 58 into an ionized vapor layer 312 or plasma. As a result of the applied voltage difference between electrode terminals 58 and the target tissue 302 (i.e., the voltage gradient across the plasma layer 312), charged particles 315 in the plasma (e.g., electrons) are accelerated towards the tissue. At sufficiently high voltage differences, these charged particles 315 gain sufficient energy to cause dissociation of the molecular bonds of tissue components. This molecular dissociation is accompanied by the volumetric removal (i.e., ablative sublimation) of tissue and the production of low molecular weight gases 314, such as oxygen, nitrogen, carbon dioxide, hydrogen and methane. The ablation of target tissue by this process can be precisely controlled, thereby avoiding or minimizing damage to adjacent non-target tissue.

In some embodiments, the voltage difference will be sufficient to apply thermal energy to the underlying tissue 320. Preferably, this thermal energy will be sufficient to elevate the tissue temperature from normal body temperatures (e.g., 37° C.) to temperatures in the range of 45° C. to 90° C., preferably in the range from 55° C. to 70° C. In the case of certain dermatological procedures, the target tissue is typically exposed to a temperature in the range of from about 55° C. to 62° C. A temperature within this range causes contraction of the collagen fibers of the skin. The invention can remove the surface layer of the skin, while, in the same procedure, tightening the underlying dermis to remove wrinkles and rejuvenate the skin.

An alternative method for skin rejuvenation or wrinkle removal is shown in FIG. 17. In this method, when a voltage difference is applied between the electrode terminals 58 and the return electrodes 100, 102, electrical current flows between the 10 electrode terminals 58 and the return electrode 100, 102 along current flux lines 350. The current flux lines 350 flow a short distance, L₄ into the surface of epidermal tissue 352 and through the electrically conductive fluid 354, in the region above the surface of the tissue, to complete the electrical path between the electrode terminals 58 and the return electrodes 100, 102. As a consequence of the electrical impedance of the tissue and the proper selection of the applied frequency, voltage, and current, heating of the epidermal tissue 352 occurs in a region 360 below the surface of the tissue 352. This heating elevates the temperature of the tissue and separates the epidermal tissue layer 352 from the underlying papillary dermis 362. The epidermal tissue layer 352 may then be removed by flushing the treatment site, or by brushing away this tissue layer 352 with, for example, a cloth pad, gauze, etc. In skin rejuvenation procedures, collagen may be injected into the dermis after the epidermis has been removed to rejuvenate skin that has lost its elasticity.

In addition, the heating from current flux lines 350 may be sufficient to elevate the temperature of the tissue 364 in the papillary dermis 362 from normal body temperature (e.g. 37° C.) to a temperature in the range 55° C. to 85° C., preferably in the range from 60° C. to 70° C. This heating of the papillary dermis 362 will cause irreversible contraction of the collagen fibers within the papillary dermis.

FIGS. 18 and 19 illustrate a method for treating a vascular lesion, such as a port wine stain, face vein, birth mark, or the like. As shown in FIG. 18, an electrosurgical probe 370 is placed on or adjacent to the surface of the skin 372 above the vessel 374 to be treated. A voltage difference is applied between the active and return electrodes (not shown) in the presence of electrically conductive fluid 376 to ablate or cause molecular dissociation of the tissue adjacent the probe 370. As the tissue is removed, the probe is axially translated through the deepening hole to the vessel 374 (note that a substantially linear probe shaft is preferred in this embodiment). A more complete description of systems and methods for forming channels or holes through tissue is described in commonly assigned, U.S. Pat. No. 5,683,366, the complete disclosure of which is incorporated herein by reference. Once the probe approaches the vessel, thermal energy is delivered into the vessel from the current flux lines as described above. This thermal energy will eventually be sufficient to coagulate the blood in the vessel 374 and collapse the vessel at that site.

In order to collapse a long length of the vessel 374, multiple treatment sites may be necessary. As shown in FIG. 19, it is desirable to locate the first treatment site 380 at a downstream point with respect to the flow of blood in the vessel. The surgeon may then sequentially treat the vessel at multiple sites (382, 384, 386) upstream from the first site 380.

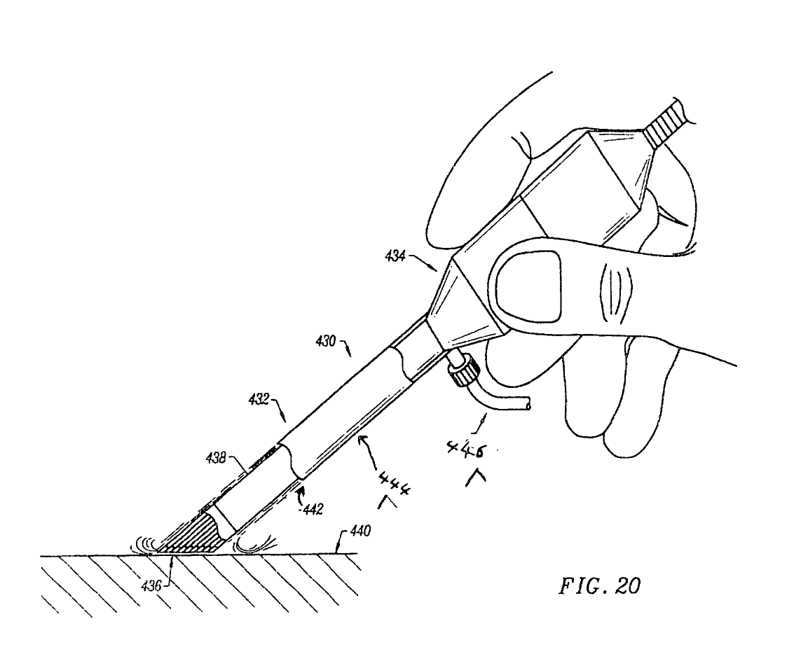

FIG. 20 illustrates an alternative embodiment, where an electrosurgical probe 430 is utilized to remove the surface layers of the epidermis 440. Probe 430 includes a shaft 432 coupled to a proximal handle 434 for holding and controlling shaft 432. Probe 430 includes an active electrode array 436 at the distal end of shaft 432, an annular return electrode 438 extending through shaft 432 and located proximal to active electrode array 436, and an annular lumen 442 between return electrode 438 and an outer insulating sheath 444. Probe 430 further includes a liquid supply conduit 446 attached to handle 434 and in fluid communication with lumen 442 and a source of electrically conductive fluid (not shown) for delivering the fluid past return electrode 438 to the target site on the epidermis 440. As discussed above, electrode array 436 is preferably flush with the distal end of shaft 432, or distally extended from the distal end by a small distance (on the order of 0.005 inches), so as to minimize the depth of ablation. Preferably, the distal end of shaft 432 is beveled to improve access and control of probe 430 while treating the target tissue, e.g., the skin.

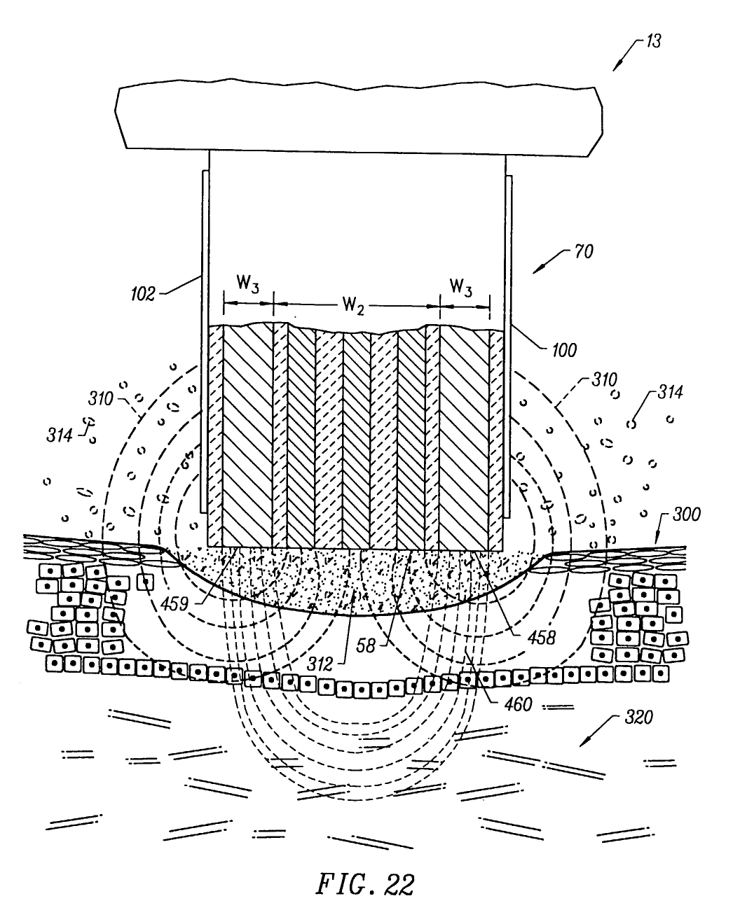

Yet another embodiment of the present invention is shown in FIG. 22. This embodiment is similar to that shown in FIGS. 16A, 16B and described above, with the exception that additional electrode terminals 458, 459 are positioned at the distal end 70 of the probe. Electrode terminals 458, 459 may be the same size as ablation electrode. terminals 58, or terminals 458, 459 may be larger than terminals 58, as shown in FIG. 22. One operating arrangement is to connect electrode terminals 458, 459 to two poles of a high frequency generator to form a bipolar circuit allowing current to flow between terminals 458, 459 as shown by current flux lines 460. The electrode terminals 458, 459 are electrically isolated from ablation electrodes 58. By proper selection of the inter-electrode spacing, W₂, the electrode width, W₃, and the frequency of the applied voltage, the current flux lines 460 can be caused to flow below the epidermis layer to effect collagen shrinkage in region 320 as described hereinabove.

The applied voltage will preferably be sufficient to establish high electric field intensities between the active electrode array and the epidermal tissue 320 to thereby induce molecular breakdown or disintegration of several cell layers of the epidermal tissue. As described above, a sufficient voltage is applied to terminals 58 to develop a thin layer of vapor within the electrically conductive fluid and to ionize the vaporized layer or region between the active electrode terminal(s) 58 and the target tissue 300. The extent, (e.g., depth) to which epidermal tissue is ablated can be precisely controlled, thereby avoiding or minimizing damage to the underlying dermis.

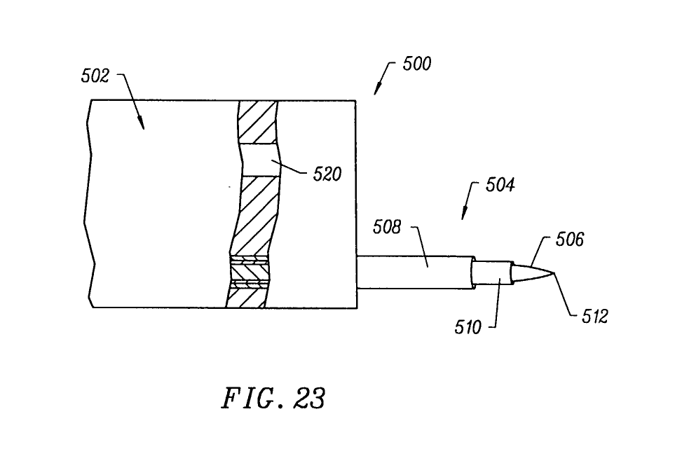

FIG. 23 illustrates a distal portion of another electrosurgical probe 500 according to the present invention, particularly useful for cutting or creating incisions in an external skin surface. Probe 500 comprises a support member 502 coupled to a shaft or disposable tip (not shown) as described in previous embodiments. Support member 502 typically comprises an electrically insulating material, such as a ceramic, a glass, or a glass-ceramic. In this embodiment, however, support member 502 may comprise an organic material, such as plastic, because the active electrode 506 and return electrode 508 are both spaced away from support member 502. Thus, the high intensity electric fields may be far enough away from support member 502 so as to allow an organic material.

An electrode assembly 504 extends from a distal end of support member 502, preferably by a distance in the range of from about 2 to 20 mm. Electrode assembly 504 comprises a single, active electrode 506 and a return electrode 508 in the form of a sleeve, and spaced proximally from active electrode 506 by an insulation member 510, which preferably comprises an inorganic material, such as a ceramic, a glass or a glass-ceramic. As shown, active electrode 506 preferably tapers to a sharp distal end 512 to facilitate the cutting or incising of tissue. In the exemplary embodiment, active electrode 506 has a proximal diameter of about 0.2 to 20 mm, and a distal diameter of less than about 0.2 mm. Return electrode 508 is spaced from active electrode 506 a sufficient distance to prevent shorting or arcing therebetween at voltages sufficient to allow the volumetric removal of tissue. In the representative embodiment, the distal end of return electrode 508 is spaced about 0.5 to about 5 mm from the proximal exposed portion of active electrode 506. Of course, it will be recognized that the present invention is not limited to the particular dimensions and configuration of the electrode assembly 504 described herein, and a variety of different embodiments may be envisioned depending on the surgical application.

As shown, probe 500 includes a fluid lumen 520 passing through support member 502 to a distal opening (not shown) at the distal end of support member 502. Fluid lumen 520 is coupled to a supply of electrically conductive fluid, such as isotonic saline, or other suitable conductive fluid for delivery of such fluid to the target site. In the exemplary embodiment, the probe is designed such that lumen 520 is positioned above electrode assembly 504 during use of probe 500, such that the conductive fluid exiting the distal opening of lumen 520 will naturally pass over return electrode 508 and active electrode 506 thereby creating a current path therebetween. In addition, the conductive fluid will be sufficient to cover active electrode 506 such that the conditions for plasma formation can be met, as described in detail above.

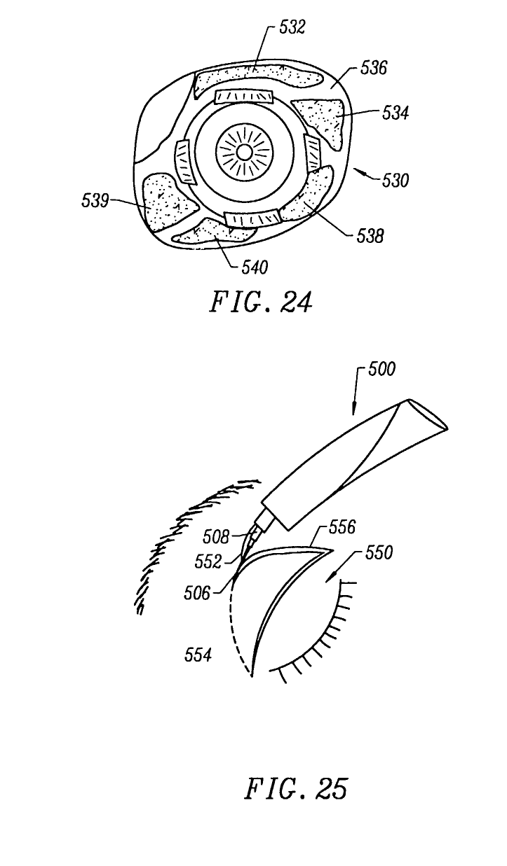

Referring now to FIGS. 24-26, a blepharoplasty procedure for removing fatty tissue underlying a patient's eyelids will now be described according to the present invention. As shown in FIG. 24, a front view of the orbit of the eye 530 reveals the important periocular structures relevant to blepharoplasty surgery. As shown, the two fat compartments of the upper lid, the central and medial compartments 532, 534, are divided by the superior oblique muscle 536. The inferior orbital fat is divided into three compartments, the medial compartment 538, the lateral fat compartment 539 and the central fat compartment 540. Medial fat has more blood vessels and nerves than the other fat compartments in both the upper and lower eyelid. Accordingly, this fat is more sensitive to the application of energy in conventional systems. Depending on the particular procedure, the present invention is designed to facilitate access to these fat compartments of the upper and lower eyelids such that a portion of the fat therein can be removed to treat “baggy eyelids” syndrome.

As shown in FIG. 25, the electrosurgical probe 500 is positioned adjacent the target area, in this case the patient's upper eyelid 550. The power supply is activated such that a high frequency voltage difference is applied between the active and return electrodes 506, 508, and electrically conductive fluid 552 is delivered to the target area, either by gravity, pump or other means. The surgeon then positions the tip of active electrode 506 adjacent to, or in contact with, the external surface of the skin 554, and translates the tip across the upper eyelid 550 to form an incision 556 therein. As discussed previously, the high frequency voltage is sufficient to convert the electrically conductive fluid between the target tissue and active electrode 506 into an ionized vapor layer or plasma. As a result of the applied voltage difference between active electrode 506 and the target tissue (i.e., the voltage gradient across the plasma layer), charged particles in the plasma (viz., electrons) are accelerated towards the tissue. At sufficiently high voltage differences, these charged particles gain sufficient energy to cause dissociation of the molecular bonds within tissue structures. This molecular dissociation is accompanied by the volumetric removal (i.e., ablative sublimation) of tissue and the production of low molecular weight gases, such as oxygen, nitrogen, carbon dioxide, hydrogen and methane.

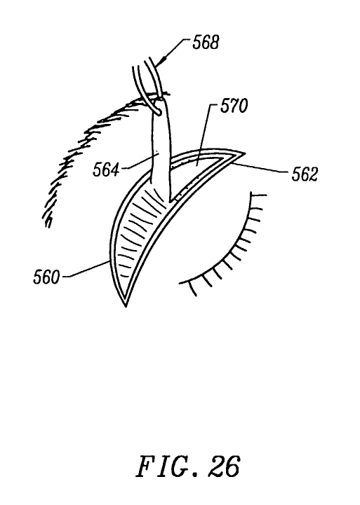

As shown in FIG. 26, the surgeon will typically create an upper incision line 560 and a lower incision line 562 to form a crescent shaped flap of skin 564 between the two incision lines 560, 562. The flap of skin 564 is then removed, either completely or by folding it over with a pair of forceps 568, to expose the underlying orbital septum 570. The orbital septum 570 is then pierced with the electrosurgical probe of the present invention or with conventional tools, such as a scalpel, and the underlying fat is excised, e.g., with forceps or other conventional tools. During excision of fat, the probe 500 may be used to effect hemostasis of any severed blood vessels at the target site. Once the desired amount of fat tissue has been removed, the surgeon reattaches the flap of skin 564 by closing the incisions.

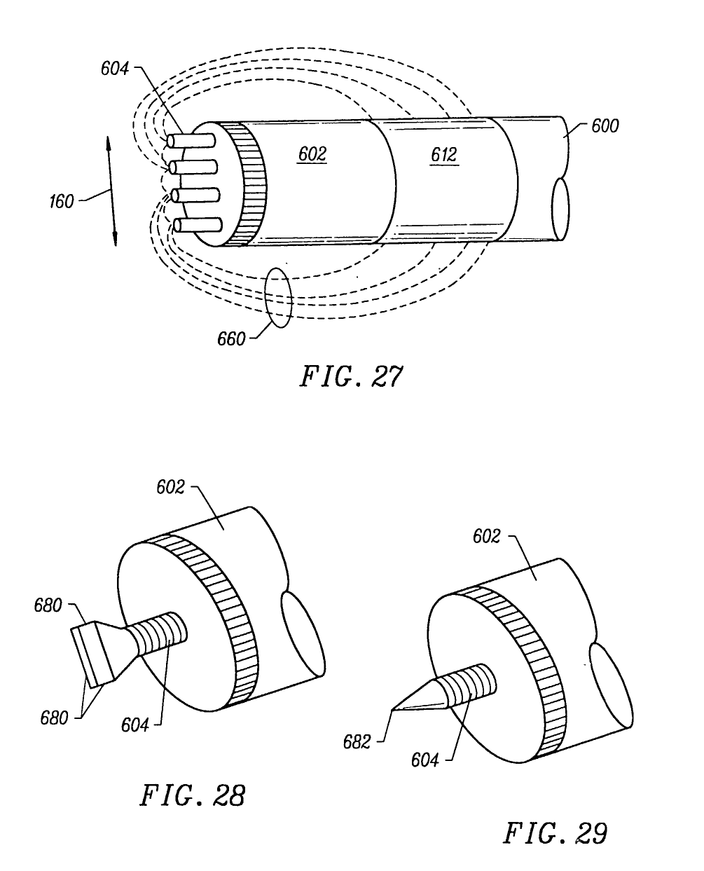

Other modifications and variations can be made to disclose embodiments without departing from the subject invention as defined in the following claims. For example, FIG. 27 illustrates yet another embodiment designed for cutting of body structures, particularly creating incisions in external skin surfaces. In this embodiment, the electrode terminals 604 are arranged in a linear or columnar array of one of more closely spaced columns so that as the electrodes 604 are moved along the longer axis (denoted by arrow 160 in FIG. 27), the current flux lines are narrowly confined at the tip of the electrode terminals 604 and result in a cutting effect in the body structure being treated. As before, the current flux lines 660 emanating from the electrode terminals 604 pass through the electrically conductive liquid to the return electrode structure 612 located proximal to electrode terminals 604.

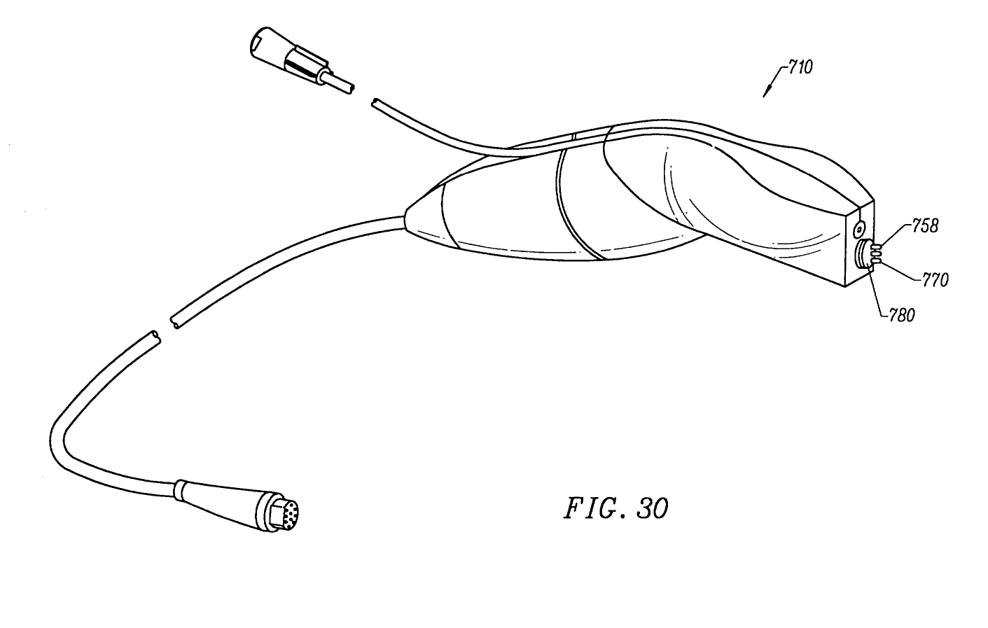

Referring now to FIGS. 28 and 29, alternative geometries are shown for the electrode terminals 604. These alternative electrode geometries allow the electrical current densities emanating from the electrode terminals 604 to be concentrated to achieve an increased ablation rate and/or a more concentrated ablation effect due to the fact that sharper edges (i.e., regions of smaller radii of curvature) result in higher current densities. FIG. 28 illustrates a flattened extension of a round wire electrode terminal 604 which results in higher current densities at the edges 680. Another example is shown in FIG. 29 in which the electrode terminal 604 is formed into a cone shaped point 682 resulting in higher current densities at the tip of the cone.

FIG. 30 illustrates yet another embodiment of a probe 710 designed for cutting or incising tissue. As shown, in the embodiment, the electrically isolated electrode terminals 758 are spaced apart over a tissue treatment surface 780 of the electrode support member 770, preferably in a linear array. In the representative embodiment, three electrode terminals 758, each having a substantially conical shape, are arranged in a linear array extending distally from surface 780. Electrode terminals 758 will usually extend a distance of about 0.5 to 20 mm from tissue treatment surface 780, preferably about 1 to 5 mm. Applicant has found that this configuration increases the electric field intensities and associated current densities at the distal edges of electrode terminals 758, which increases the rate of tissue cutting. In the representative embodiment, the tissue treatment surface 780 is circular or substantially circular, with a diameter in the range of from about 0.5 mm to 20 mm (preferably about 2 to 10 mm). The individual electrode terminals 758 preferably taper outward as shown, or they may form a distal edge, such as the electrodes shown in FIG. 28.

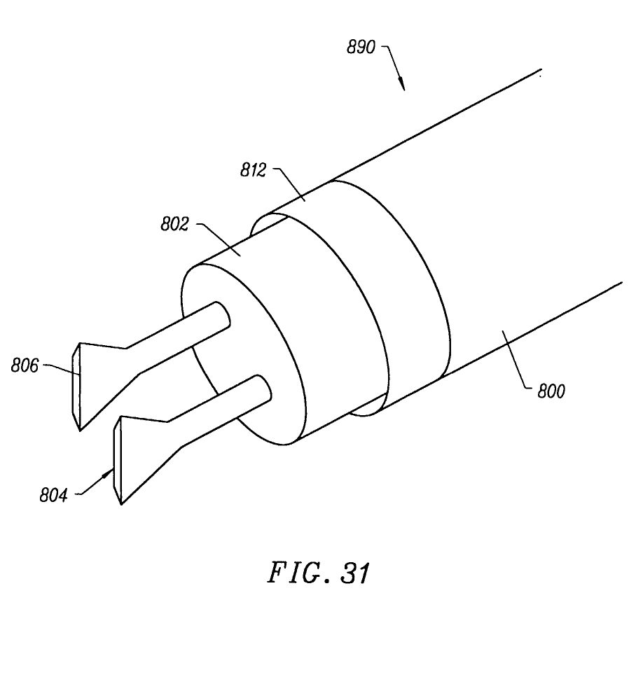

FIG. 31 illustrates an electrosurgical probe 890 comprising a shaft 800 and at least two electrode terminals 804 extending from a support matrix 802 at the distal end of the shaft. The electrode terminals 804 preferably define a distal edge 806 for cutting an incision in tissue. The edges 806 of the electrode terminals 804 are substantially parallel with each other, and usually spaced apart by a distance of about 4 to 15 mm, preferably about 8 to 10 mm. The edges 806 extend from the distal end of support matrix 802 by a distance of about 0.5 to 10 mm, preferably about 2 to 5 mm. In the exemplary embodiment, probe 890 will include a return electrode 812 spaced proximally from the electrode terminals 804. Alternatively, the return electrode 812 may be one of the electrode terminals 804, or it may be a dispersive pad located on an external surface of the patient's body.

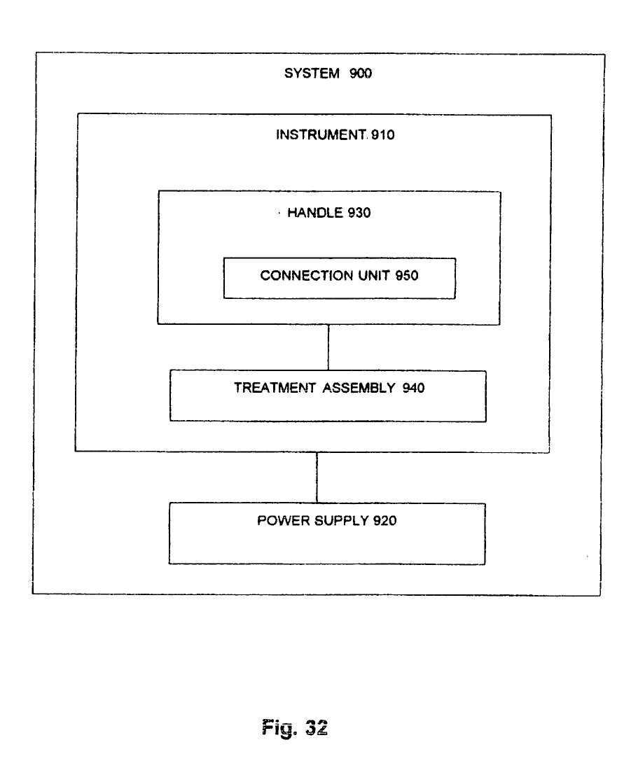

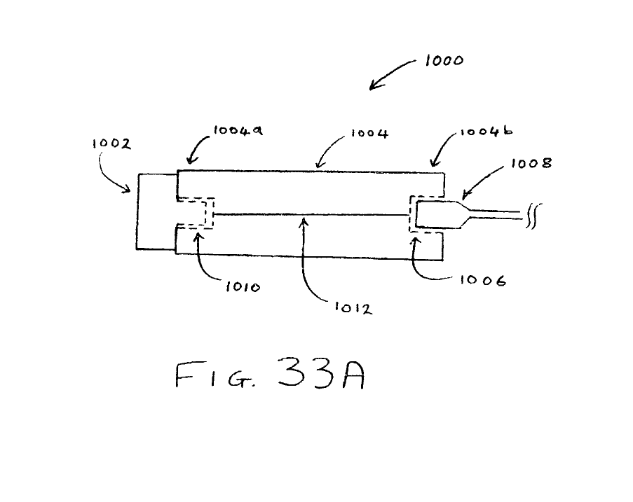

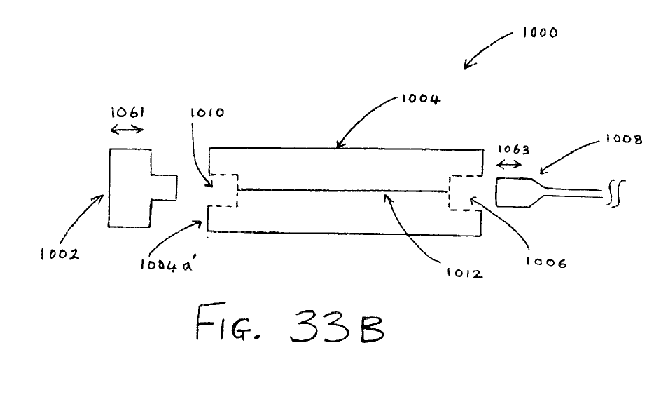

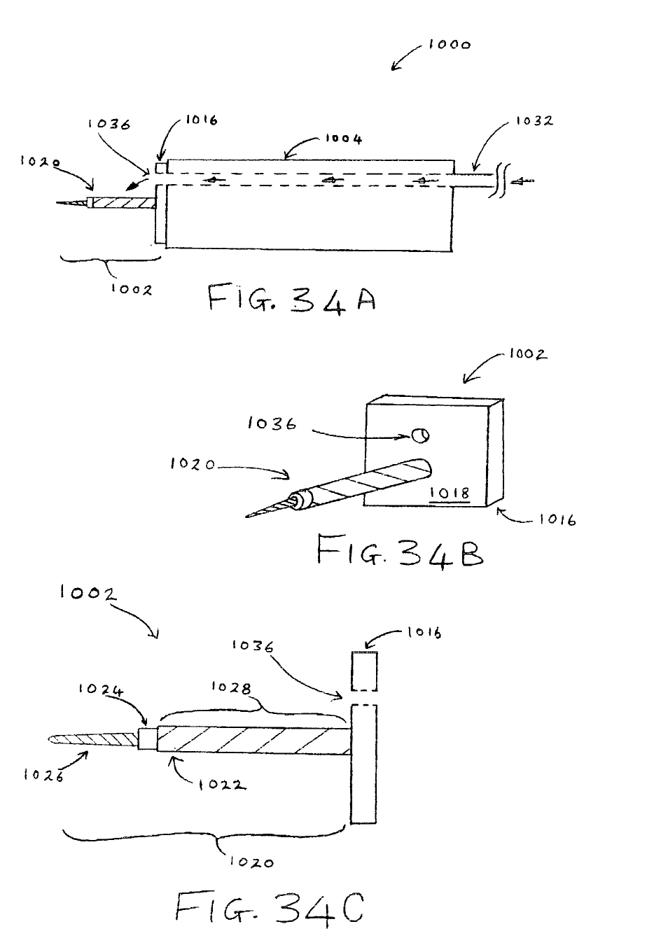

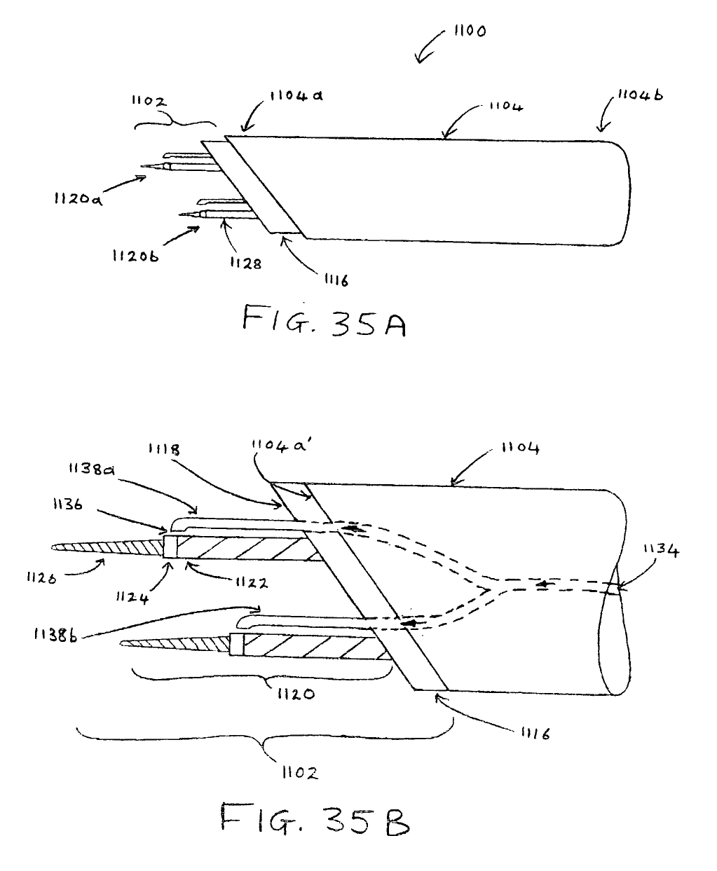

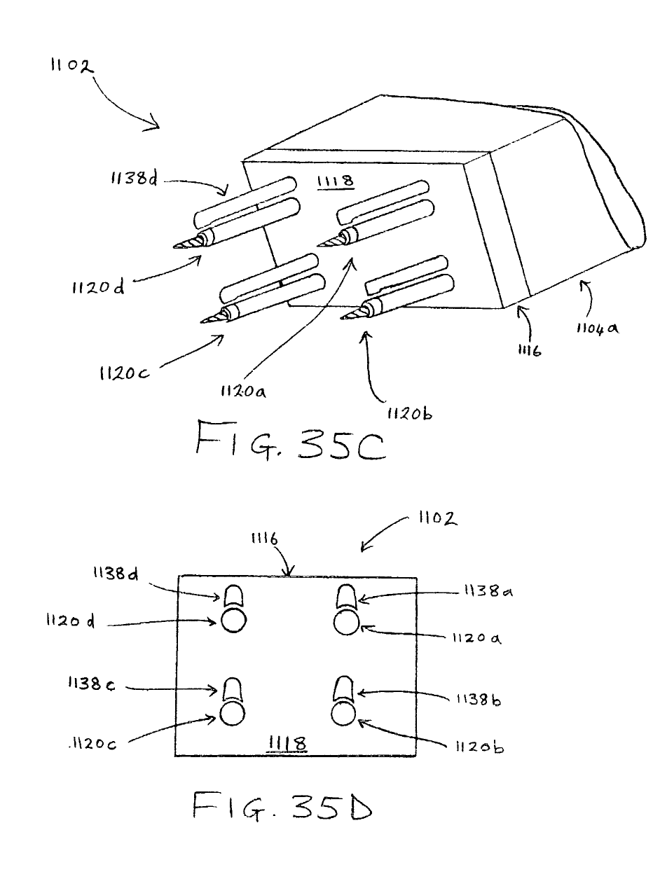

FIG. 32 is a block diagram representing an electrosurgical system 900, according to another embodiment of the present invention. System 900 generally includes an electrosurgical instrument 910, electrically coupled to a high frequency power supply 920. Instrument 910 includes a handle 930, a treatment assembly 940, and a connection unit 950. Connection unit 950 may include a first connection block adapted for electrically coupling handle 930 to power supply 920, and a second connection block adapted for electrically coupling treatment assembly 940 to handle 930 (e.g., FIGS. 33A, 33B). Treatment assembly 940 includes one or more active electrode terminals disposed on one or more treatment units (e.g., FIGS. 34A-C, FIGS. 35A-C). Typically, system 900 is configured and adapted for the controlled and selective ablation of tissue, for example skin tissue, from a target site of a patient. Preferably, such ablation occurs via plasma-induced molecular dissociation of tissue components at a temperature in the range of from about 45° to 90° C., substantially as described hereinabove.

FIG. 33A shows an electrosurgical instrument 1000, according to one embodiment of the instant invention. Instrument 1000 includes a hand-piece or handle 1004, and a distal treatment assembly 1002. Handle 1004 includes a handle distal end 1004a and a handle proximal end 1004b. Typically, treatment assembly 1002 is removably attached to handle distal end 1004a (e.g., FIG. 33B). Handle 1004 typically comprises a housing, which houses a proximal connection block 1006 and a distal connection block 1010. Proximal connection block 1006 is adapted for conveniently coupling handle 1004 to power supply 920 via a connector cable 1008. Typically, connector cable 1008 comprises an electrical cable, substantially as described with reference to FIG. 1. Distal connection block 1010 is adapted for conveniently coupling treatment assembly 1002 to handle 1004. Proximal connection block 1006 and distal connection block 1010 may be connected by coupling lead 1012. In one embodiment, electrical connections between proximal connection block 1006 and treatment assembly 1002 may be made in a manner analogous to that described hereinabove, e.g., with reference to FIG. 5.

FIG. 33B shows electrosurgical instrument 1000 in a disassembled condition, i.e., with treatment assembly 1002 detached from handle 1004. Handle 1004 includes a handle distal face 1004a′. In one embodiment, handle distal face 1004a′ is configured as a substantially square or rectangular plane, although other configurations are also within the scope of the invention. Typically, treatment assembly 1002 is electrically coupled to handle 1004 via distal connection block 1010, and affixed to handle 1004 at handle distal face 1004a′. Attachment and detachment of treatment assembly 1002 to/from handle distal end 1004a is indicated by the double-headed arrow 1061. Attachment and detachment of connector cable 1008 to/from handle proximal end 1004b is indicated by the double-headed arrow 1063. In one embodiment, handle 1004 is sterilizable and may be reused, i.e., may be used in a number of separate procedures. In one embodiment, handle 1004 comprises a durable, heat-resistant, or refractory material that can withstand repeated exposure to heat. In one embodiment, handle 1004 can be autoclaved repeatedly without detracting from the functionality of handle 1004. Treatment assembly 1002 is typically disposable after a single use. In another embodiment, both handle 1004 and treatment assembly 1002 are single-use (disposable) items. In a further embodiment, both handle 1004 and treatment assembly 1002 are sterilizable and re-usable.

FIG. 34A schematically represents an electrosurgical instrument 1000′, according to the invention. Instrument 1000′ includes handle 1004 and treatment assembly 1002 disposed at the distal end of handle 1004. Treatment assembly 1002 includes a treatment base 1016, and a treatment unit 1020 extending distally from base 1016. Treatment assembly 1002 is attached to handle 1004 via base 1016. Instrument 1000′ further includes a fluid delivery unit comprising a fluid delivery lumen 1034 within handle 1004, a proximal fluid delivery tube 1032, and a distal fluid delivery port 1036. Fluid delivery port 1036 is configured to deliver an electrically conductive fluid (as represented by the solid arrows) to treatment unit 1020 during use of instrument 1000′.

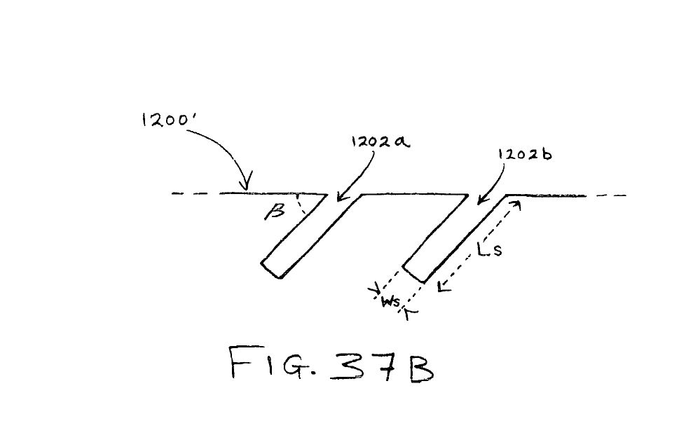

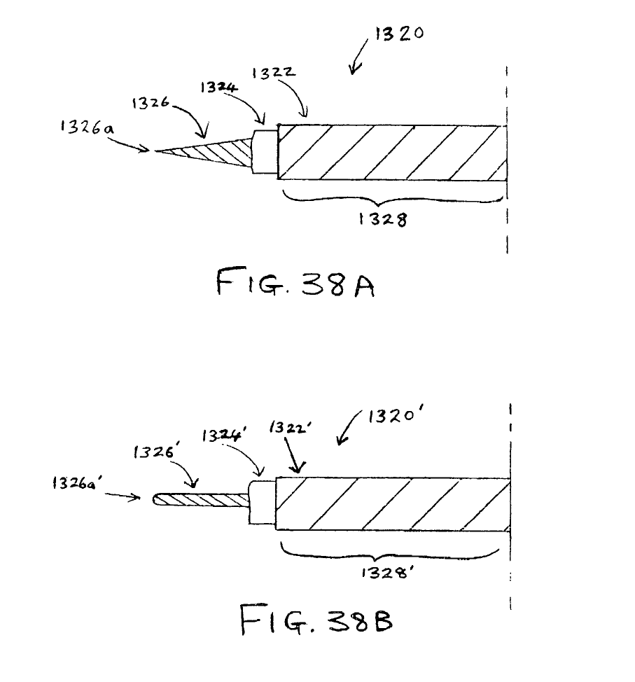

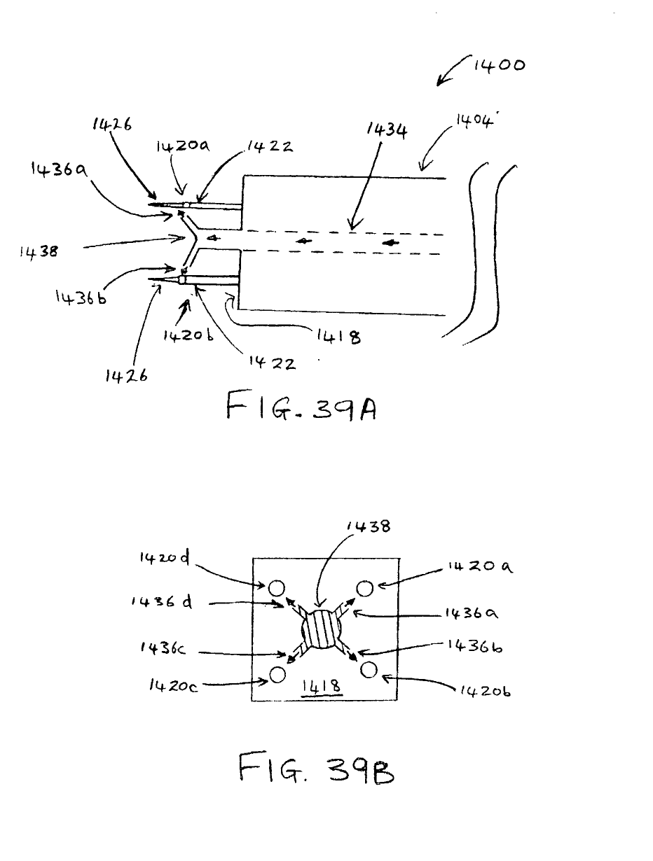

FIG. 34B is a perspective view of treatment assembly 1002, showing a treatment surface 1018 of base 1016. In one embodiment, base 1016 is adapted for conveniently attaching and detaching treatment assembly 1002 to and from the distal or working end of handle 1004. For example, base 1016 may be conveniently attached to handle 1004 via one or more snap connectors, well known in the art. Treatment unit 1020 may be electrically coupled to handle 1004 via a distal connection block (e.g., block 1010, FIGS. 33A-B). As shown, fluid delivery port 1036 is in the form of an opening in base 1016 located superior to treatment unit 1020. However, other configurations for fluid delivery port(s) are also within the scope of the invention (e.g., FIGS. 39A-B, FIGS. 43A-C). In one embodiment, a treatment assembly may include a plurality of treatment units and a plurality of fluid delivery ports.

FIG. 34C is a side view of treatment assembly 1002 showing details of treatment unit 1020, according to one embodiment of the invention. Treatment unit 1020 includes a shaft 1028 extending from base 1016, an electrode support 1024 disposed on shaft 1028, a return electrode 1022 located proximal to electrode support 1024, and an active electrode terminal 1026 disposed on electrode support 1024. Typically, electrode support 1024 comprises an electrically insulating material, such as a ceramic, a glass, or a silicone rubber. Shaft 1028 may comprise an electrically conducting material, wherein an external surface of shaft 1028 serves as return electrode 1022. For example, shaft 1028 may comprise stainless steel, or other metals, or their alloys, and the like. Alternatively, the shaft may be partially encased within an electrically insulating sleeve (not shown in FIG. 34C), wherein an exposed distal portion of the shaft serves as a return electrode. In yet another embodiment (also not shown), the shaft may comprise an electrically insulating material, such as various synthetic polymers, and the return electrode may comprise an annular band of an electrically conducting material disposed on the exterior of the shaft.

FIG. 35A schematically represents an electrosurgical instrument 1100, according to another embodiment of the invention. Instrument 1100 includes a handle 1104 having a beveled distal end 1104a, and a distal treatment assembly 1102. Treatment assembly 1102 includes a treatment base 1116, having a plurality of treatment units 1120a-d and a plurality of fluid delivery channels emanating from base 1116 (e.g., FIGS. 35C, 35D).

FIG. 35B is a side view of the distal portion of instrument 1100 showing a fluid delivery lumen 1134 within handle 1104. Fluid delivery lumen 1134 branches to provide electrically conductive fluid to each of the plurality of fluid delivery channels, 1138a-d (FIG. 35C).

FIGS. 35C and 35D show a perspective view, and an end view, respectively, of the distal portion of instrument 1100 of FIG. 35A. Treatment units 1120a-d extend distally from beveled treatment surface 1118. With reference to FIGS. 35A-D, each treatment unit 1120a-d includes a shaft 1128, an electrode support 1124 disposed on the distal portion of shaft 1128, an active electrode terminal 1126 disposed on electrode support 1124, and a return electrode 1122 located proximal to electrode support 1124. Each active electrode terminal 1126 is adapted for forming a void, or graft socket, in the skin. The length and width (or diameter) of each active electrode terminal 1126 is generally within the range given hereinabove for electrode terminals of other embodiments of the invention. For example, each active electrode terminal 1126 may have a length in the range of from about 1 mm to about 10 mm, and a width (or diameter) in the range of from about 0.1 to about 3 mm.

As shown, base 1116 is attached to handle distal face 1104a′ (FIG. 35B). In one embodiment, treatment assembly 1102 may be removably attached to handle 1104, wherein treatment assembly 1102 is disposable, and handle 1104 is adapted for sterilization and re-use, essentially as described hereinabove with reference to FIG. 33B. Each active electrode terminal 1126 and return electrode 1122 of treatment assembly 1102 may be independently coupled to power supply 920, for example, via a connection unit (e.g., FIG. 32).

With reference to FIGS. 35C and 35D, a fluid delivery channel 1138a-d, is arranged adjacent to each treatment unit 1120a-d. For example, fluid delivery channel 1138a lies adjacent to treatment unit 1120a. The distal portion of each fluid delivery channel 1138a-d may be somewhat curved towards its corresponding treatment unit 1120a-d, such that electrically conductive fluid delivered from each fluid delivery port 1136 is directed towards the distal end of each treatment unit 1120a-d. The electrically conductive fluid delivered to the distal end of each treatment unit 1120a-d provides a current flow path between active electrode terminal 1126 and return electrode 1122 of each treatment unit 1120a-d.

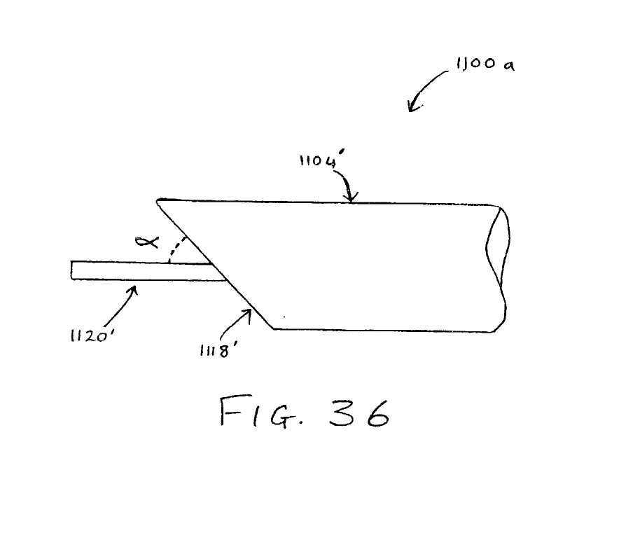

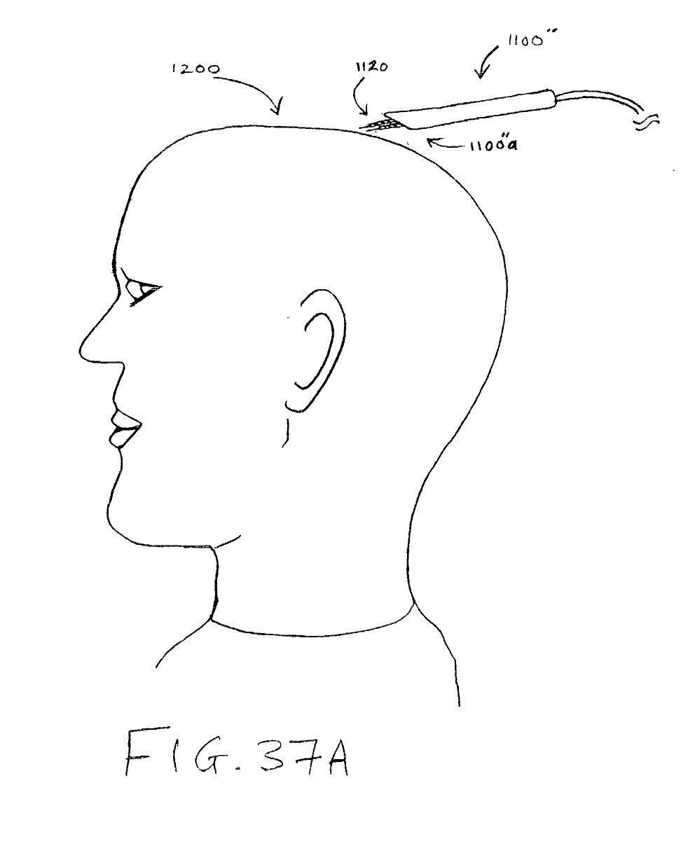

FIG. 36 is a side view of the distal portion 1100′a of an electrosurgical instrument having a handle 1104′, a beveled treatment surface 1118′, and a treatment unit 1120′ arranged at an angle ∀ to treatment surface 1118′. The bevel of treatment surface 1118′, and the arrangement of treatment unit 1120′ at angle ∀ to treatment surface 1118′, facilitate positioning of treatment unit 1120′ with respect to a target tissue during certain procedures, such as forming voids, or sockets, in the scalp of a patient (e.g., FIGS. 37A, 37B). Angle ∀ is typically an acute angle in the range of from about 5° to 85°.

FIG. 37A schematically represents formation of a plurality of small voids, or graft sockets, in the scalp of a patient during an autologous hair transplant procedure. An electrosurgical instrument 1100″ is positioned such that the distal end 1100″a is in at least close proximity to a recipient area of the scalp 1200 of a patient. By way of example, a recipient area may be an area of skin which is to receive one or more follicular grafts. Instrument 1100″ includes at least one treatment unit 1120. Each treatment unit 1120 typically includes an active electrode terminal (e.g., terminal 1126, FIG. 35B). Typically, instrument 1100″ includes a plurality of treatment units 1120. In one embodiment, a plurality of treatment units 1120 are arranged in a square configuration, for example, in a 2×2 array (e.g., FIGS. 35c, 35D). In other embodiments, a plurality of treatment units 1120 may be arranged in a 3×3 array, or a 4×4 array, to provide nine and 16 treatment units 1120 per array, respectively. Embodiments having even larger numbers of treatment units per instrument are also within the scope of the invention. Of course, in certain procedures, or stages in a procedure, an electrosurgical instrument having a single treatment unit and a single active electrode terminal may be used for forming graft sockets in the scalp 1200, or other regions of the skin.

In use, instrument 1100″ is coupled to a suitable power supply (e.g., FIG. 1, FIG. 32) to provide a high frequency voltage to each active electrode terminal of instrument 1100″. Each active electrode terminal of instrument 1100″ is adapted for forming a void, in the form of a graft socket, in skin tissue (e.g., FIG. 37B). The beveled nature of instrument distal end 1100″ a facilitates positioning treatment units 1120 in at least close proximity to the scalp 1200, and in forming voids (sockets) at various angles with respect to the surface of the skin of the patient (e.g., FIG. 37B). Naturally, other configurations for the distal end of the electrosurgical instrument are also possible under the invention.

FIG. 37B shows a plurality of graft sockets 1202a, 1202b formed in the skin 1200′, according to one embodiment of the invention. Graft sockets 1202a, 1202b each represents a void within the skin 1200′ formed by the localized volumetric removal of skin tissue upon application of high frequency voltage to each treatment unit 1120 (e.g., FIG. 37A). During a hair transplant procedure, each graft socket, e.g., 1202a, accommodates a follicular graft, as is described in detail hereinbelow. The size of the graft sockets may be tailored to suit follicular grafts of a particular size and shape. In general, the size of the graft sockets is determined by the shape and size of the treatment units 1120, the applied voltage, the duration of treatment, and the nature of any manipulation of the instrument by the surgeon, etc. Typically, the graft sockets 1202a, 1202b have a length Ls in the range of from about 0.5 mm to about 3.0 mm, and a width (or diameter) Ws in the range of from about 0.5 mm to about 2.0 mm. In one embodiment, the graft sockets 1202a, 1202b are formed at an angle ∃ to the surface of the skin 1200′. Typically, angle ∃ is in the range of from about 5° to about 80°. Arrangement of implanted follicular grafts at a suitable angle to the scalp 1200 generally provides a more natural appearance to the transplanted hair. Although graft sockets 1202a, 1202b are represented in FIG. 37B as having substantially parallel sides, sockets having other shapes are also contemplated under the invention.

FIGS. 38A and 38B each show a side view of a treatment unit, 1320, 1320′, respectively, according to two different embodiments of the invention. Treatment unit 1320 of FIG. 38A includes an electrode support 1324 disposed on a shaft 1328, an active electrode terminal 1326 extending distally from electrode support 1324, and a return electrode 1322 located proximal to electrode support 1324. Active electrode terminal 1326 is substantially conical, and terminates in a distal point or tip 1326a. In one embodiment, the return electrode may be truncated in the form of an annular band (not shown in FIG. 38A), located proximal to electrode support 1324. Alternatively, a proximal portion of shaft 1328 may be encased within an electrically insulating sleeve (also not shown), such that an exposed distal portion of shaft 1328 defines an annular return electrode.

With reference to FIG. 38B, treatment unit 1320′ includes an active electrode terminal 1326′ extending distally from an electrode support 1324′, and a return electrode 1322′ located proximal to electrode support 1324′. Active electrode terminal 1326′ is substantially cylindrical, and terminates in a less pointed distal tip 1326a′ (cf., tip 1326a, FIG. 38A).

During use of treatment unit 1320 (FIG. 38A), the pointed tip 1326a of active electrode terminal 1326 results in relatively high electric field intensities thereat, and electrode terminal 1326 is therefore generally adapted for more aggressive tissue ablation, as compared with treatment unit 1320′. Thus, treatment unit 1320 may be used for making precise incisions in skin tissue and for excising portions of the skin. The less pointed geometry of active electrode terminal 1326′ generally results in less aggressive ablation of tissue. Thus, treatment unit 1320′ may be used for the controlled ablation of tissue to form graft sockets of a pre-defined depth and width. Of course, active electrode terminals having geometries other than those of terminal 1326 and terminal 1326′, are also within the scope of the invention. The geometry of the active electrode terminal(s) may be designed so as to provide an appropriate rate of ablation, and to form a graft socket of suitable shape and dimensions to accommodate follicular grafts during a hair transplant procedure. According to one aspect of the invention, the active electrode terminal(s) may include an element or feature (not shown) to allow the surgeon to readily and accurately determine the depth of penetration of the active electrode terminal(s) into the skin tissue. For example, the active electrode terminal(s) may include one or more collars disposed at pre-defined locations with respect to the distal end of the active electrode terminal, e.g., distal tip 1326a′. Alternatively, the active electrode terminal(s) may include a movable plate that can be positioned at a specific location with respect to distal tip 1326a′. In this way, the depth of the graft sockets can be more precisely and consistently controlled.

With reference to FIGS. 39A and 39B an electrosurgical instrument 1400, according to another embodiment of the invention includes a fluid delivery lumen 1434 within a handle 1404. Fluid delivery lumen 1434 is in communication distally with a fluid delivery channel 1438, the latter emanating from a treatment surface 1418. A plurality of treatment units 1420a-d are disposed on treatment surface 1418. Each treatment unit 1420a-d includes an active electrode terminal 1426 and a return electrode 1422. Fluid delivery channel 1438 has a plurality of fluid delivery ports 1436a-d radiating therefrom. Each fluid delivery port 1436a-d is oriented towards a corresponding one of treatment units 1420a-d. For example, fluid delivery port 1436a directs electrically conductive fluid (represented by solid arrows) to the distal portion of treatment unit 1420a. In this manner, electrically conductive fluid, delivered from fluid delivery ports 1436a-d, provides a current flow path between each active electrode terminal 1426 and its corresponding return electrode 1422.

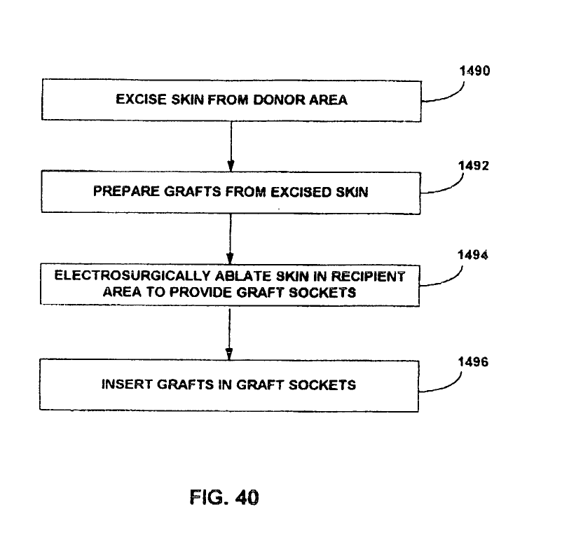

FIG. 40 schematically represents a series of steps involved in a method of performing an autologous hair transplant procedure, according to the invention, wherein step 1490 involves excising a portion of hair-bearing skin from a donor area. In one embodiment, step 1490 involves electrosurgically excising the donor skin using a suitable electrosurgical instrument (e.g., the apparatus described hereinabove with reference to FIG. 23 or FIG. 38A). For example, one or more portions of donor skin may be removed by the localized electrosurgical ablation of skin tissue to form one or more incisions in the skin. Such incisions in the skin may be made substantially as described hereinabove, e.g. with reference to FIGS. 25 and 26. During excision of donor skin in step 1490, any small blood vessels severed during the procedure may be simultaneously sealed (coagulated) due to the voltage applied to the active electrode(s) of the instrument. If necessary, the surgeon may temporarily lower the voltage from the power supply to switch to the coagulation mode (e.g., by actuating foot pedal 38, FIG. 1). The areas of donor skin excised in step 1490 may be elongate (e.g., rectangular), substantially circular, ovoid, or crescent shaped, etc. Typically, the donor area has a suitable density and quality of terminal hairs. After excision of donor skin, the donor area may be closed, e.g., by sutures.

Step 1492 involves preparing a plurality of follicular grafts from the donor skin. Typically, each follicular graft prepared from the donor skin comprises one or two hair follicles. However, larger grafts having greater numbers of follicles may also be used. Each follicular graft prepared from the donor skin can be accommodated in graft sockets of an appropriate size and shape. Step 1494 involves preparing a plurality of graft sockets in a recipient area of the skin. Each graft socket is typically formed by the localized electrosurgical ablation of skin. Typically, such ablation involves the volumetric removal of skin tissue via the plasma-induced molecular dissociation of tissue components. In this manner, a plurality of voids of suitable dimensions can be formed in the skin of the patient, wherein each void comprises a graft socket suited to receiving a follicular graft. (A method of preparing a plurality of graft sockets in recipient skin tissue, according to one aspect of the invention, is described fully hereinbelow with reference to FIG. 41.)

After step 1494, step 1496 involves inserting, or implanting, at least one of the follicular grafts in each of at least a portion of the plurality of graft sockets. The method of the invention is generally applicable to adult patients having androgenic alopecia, as well as to patients of all age groups experiencing alopecia due to factors such as emotional stress, chemotherapy, mechanical trauma, burns, or chemical injury, and the like.

FIG. 41 schematically represents a series of steps involved in a method of forming a plurality of voids, or graft sockets, in the skin of a patient. According to the invention, step 1590 involves positioning a treatment assembly of an electrosurgical instrument in at least close proximity to a target site of the patient. Typically, the target site is an external surface of the skin targeted as a recipient area for receiving follicular grafts (e.g., step 1492, FIG. 40). Usually, the target site is on the scalp. However, in a patient having suffered facial trauma, the recipient area may be the eyebrows, and, in a male patient, may include the upper lip, the sides of the face, or the chin, etc. If necessary, step 1590 may be performed under suitable magnification of the target site.

The electrosurgical instrument positioned in step 1590 may have elements, features, and characteristics of the various electrosurgical instruments or probes of the invention, as described hereinabove, e.g., with reference to FIGS. 2-5, 20, 23, 27-31, and 33A-36. Thus, the electrosurgical instrument positioned in step 1590 may include a handle having a treatment assembly attached to the handle distal end (working end), wherein the treatment assembly includes at least one treatment unit. Each treatment unit typically includes a shaft, an electrode support disposed on the shaft distal end, an active electrode terminal disposed on the electrode support, and a return electrode located proximal to the electrode support. In use, each treatment unit is electrically coupled to a high frequency power supply for applying a high frequency voltage between the active electrode terminal and the return electrode of each treatment unit.

Typically, the electrosurgical instrument positioned in step 1590 includes a fluid delivery unit having at least one fluid delivery port for supplying an electrically conductive fluid to each treatment unit. Step 1592 involves supplying an electrically conductive fluid, such as isotonic saline, to the treatment unit(s). The electrically conductive fluid provides a current flow path between the active electrode terminal and the return electrode of each treatment unit.

Step 1594 involves applying a high frequency voltage between the active electrode terminal and the return electrode of each treatment unit. The applied voltage is sufficient to cause the localized ablation of skin tissue in the vicinity of each treatment unit. The localized ablation of skin tissue forms a void or socket in the vicinity of each treatment unit, wherein the socket is suitable for accommodating a follicular graft. The voids or sockets may take various forms, such as elongate slits, cone-shaped, or cylindrical, as required by the surgeon. Typical dimensions for sockets formed according to one embodiment of the invention were described hereinabove with reference to FIG. 37B. However, sockets having other dimensions and are also within the scope of the invention.

The parameters of the voltage applied in step 1594 are typically within the ranges described hereinabove. For example, the voltage is typically applied at a frequency in the range of from about 50 kHz to 500 kHz, and more typically from about 100 kHz to 200 kHz. The root mean square (RMS) voltage is usually in the range of from about 5 volts to 1000 volts RMS, more typically from about 10 volts to 500 volts RMS, depending on the electrode terminal size and shape, and the operating frequency. Typically, the peak-to-peak voltage is in the range of from about 10 to 2000 volts, and more typically from about 40 to 800 volts (again, depending on the electrode size, and the operating frequency). Using methods of the invention, as described with reference to FIG. 41, a plurality of graft sockets can be formed rapidly, and with minimal discomfort to the patient, while at the same time avoiding or minimizing damage to surrounding non-target tissue.

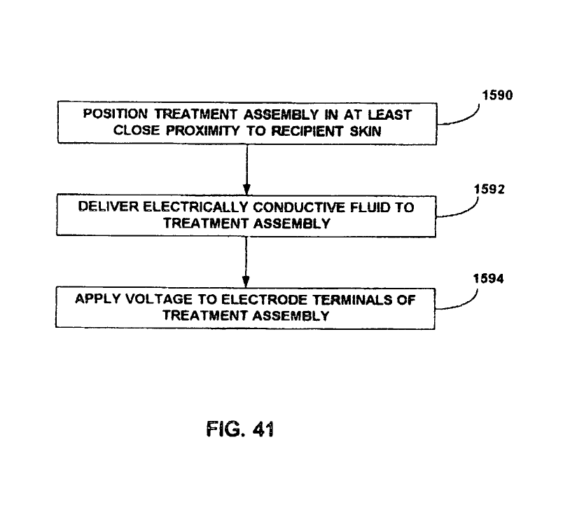

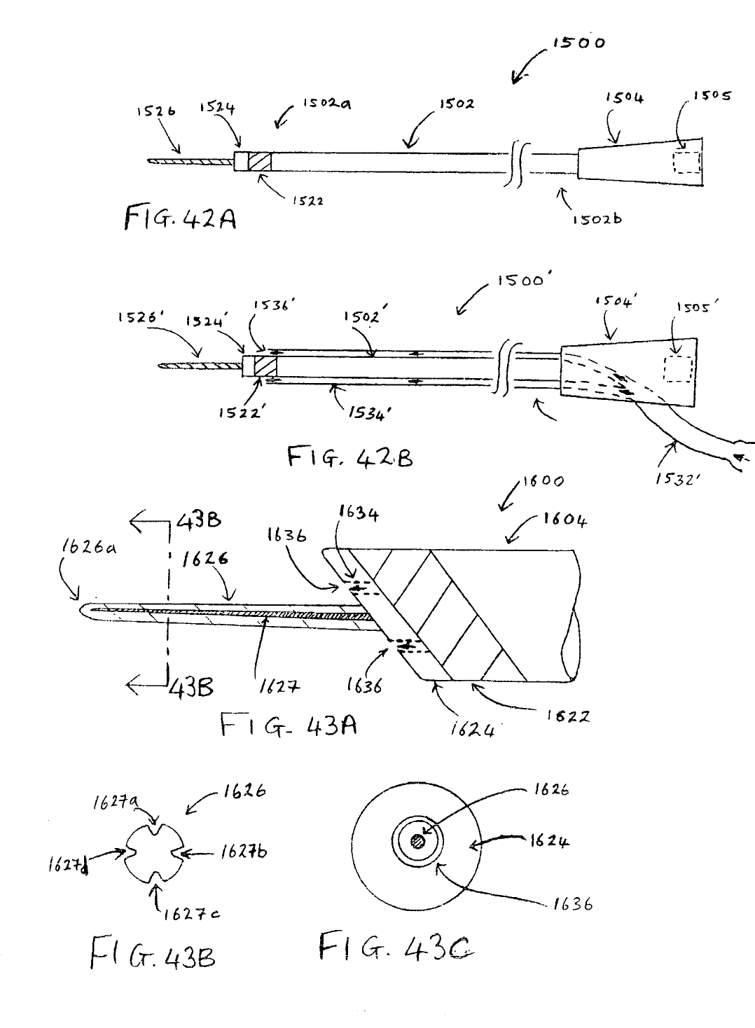

FIG. 42A is a side view of an electrosurgical probe 1500 for epilating the skin, according to another embodiment of the invention. Probe 1500 includes an elongate shaft 1502 having a shaft distal end 1502a and a shaft proximal end 1502b, and a handle 1504 affixed to shaft proximal end 1502b. An electrically insulating electrode support 1524 is disposed on shaft distal end 1502a. A single, stylet-like elongate active electrode terminal 1526 extends distally from electrode support 1524. A return electrode 1522 is disposed on shaft distal end 1502a at a location proximal to electrode support 1524. Return electrode 1522 may be spaced a sufficient distance from active electrode 1526 to minimize or substantially avoid current shorting therebetween, and to avoid contacting the target tissue with return electrode 1522.

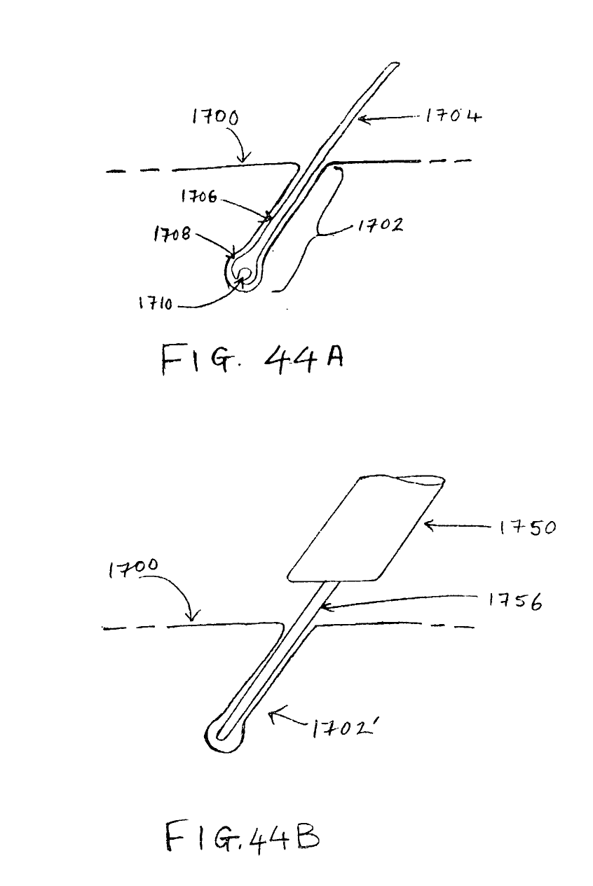

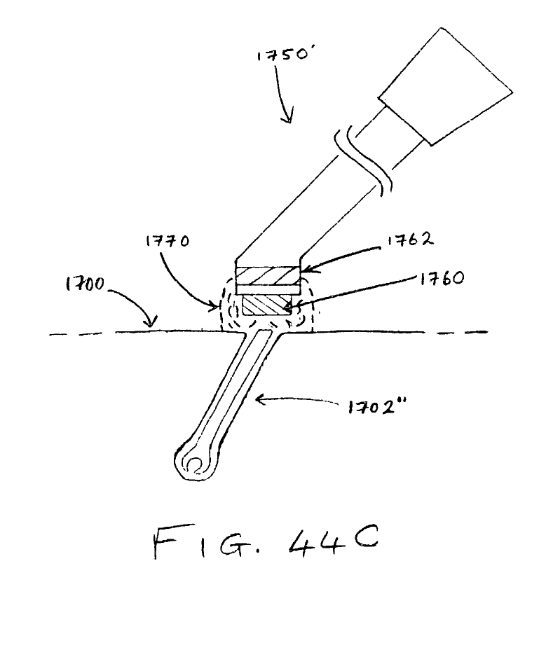

Again with reference to FIG. 42A, handle 1504 houses a connection block 1505 for conveniently coupling probe 1500 to a high frequency power supply (e.g., FIG. 1, FIG. 32). Active electrode terminal 1526 is configured for targeting individual hair follicles. Probe 1500 is adapted for the localized ablation of tissue, such as an individual hair follicle or a portion of a hair follicle, in the vicinity of active electrode terminal 1526. In one embodiment, tissue of a hair follicle, e.g., a hair bulb 1708 and/or a hair papilla 1710 (FIG. 44A), may be volumetrically removed via plasma-induced molecular dissociation of tissue components, generally at a temperature in the range of from about 45° to 90° C. In this embodiment, it is generally desirable to supply an extraneous electrically conductive fluid to the distal end of probe 1500 (e.g., FIG. 42B). The electrically conductive fluid supply promotes establishment and maintenance of a plasma layer between the active electrode terminal and the target tissue, and helps to prevent thermal damage to adjacent, non-target tissue. In an alternative embodiment, tissue of a hair follicle may be destroyed by exposing the tissue to heat, typically at a temperature above about 90° C., e.g., via the application of RF energy to the tissue.

FIG. 42B is a side view of an electrosurgical probe 1500′ for epilating the skin, according to another embodiment of the invention. Probe 1500′ includes elements and components at least somewhat analogous to those of probe 1500 of FIG. 42A. Thus, probe 1500′ includes an elongate shaft 1502′ and a handle 1504′ affixed to shaft 1502′. An electrically insulating electrode support 1524′ is disposed on the distal end of shaft 1502′. A single, elongate active electrode terminal 1526′ extends distally from electrode support 1524′, and a return electrode 1522′ is located proximal to electrode support 1524′. Probe 1500′ further includes a fluid delivery element. Thus, an external fluid delivery sheath 1534′ surrounds shaft 1502′, and terminates distally in an annular fluid delivery port 1536′. A proximal fluid delivery tube 1532′ supplies electrically conductive fluid (solid arrows) to external sheath 1534′. Electrically conductive fluid delivered from port 1536′ provides a current flow path between active electrode terminal 1526′ and return electrode 1522′.

FIG. 43A is a side view of the distal portion of an electrosurgical probe 1600 having a handle 1604, according to another embodiment of the invention. With reference to FIGS. 43A-C, an annular fluid delivery lumen 1634 is located within handle 1604. Perhaps as best seen in FIG. 43C, fluid delivery lumen 1634 terminates distally in an annular fluid delivery port 1636. An elongate active electrode terminal 1626 extends distally from an electrically insulating electrode support 1624. A return electrode 1622 is located proximal to electrode support 1624. Fluid delivery port 1636 encircles the proximal end of active electrode terminal 1626. Perhaps as best seen in FIG. 43B, active electrode terminal 1626 includes a plurality of longitudinal grooves 1627a-d therein, Grooves 1627a-d are adapted for supplying electrically conductive fluid along substantially the entire length of terminal 1626. Of course, it is to be understood that under the invention, the active electrode terminal may have alternative numbers and arrangements of grooves, other than those shown in FIGS. 43A, 43B.

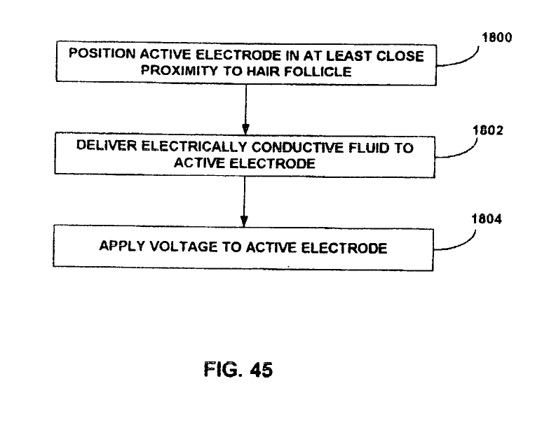

FIG. 44A represents a side view of a healthy or normal hair follicle 1702 in the skin 1700 of a patient. A hair root 1706 is shown within hair follicle 1702, and a hair shaft 1704 protrudes from the external surface of the skin 1700. In the lower portion of the hair follicle 1702, a hair papilla 1710 is capped by a hair bulb 1708. FIG. 44B shows the distal portion of an electrosurgical probe 1750 positioned with respect to the surface of the skin 1700, such that a treatment unit 1756 of probe 1750 is inserted into a hair follicle 1702′ for the purpose of ablation or destruction of at least a portion of the hair follicle 1702′. For example, hair bulb 1708 and/or hair papilla 1710 may be ablated by treatment unit 1756. In this manner, the hair shaft 1704 may be permanently removed from the skin 1700. By targeting a plurality of hair follicles in a region of the skin, the skin in that region can be epilated. A method for epilating the skin is described in detail hereinbelow (with reference to FIG. 45).

FIG. 44C shows an electrosurgical probe 1750′ positioned with respect to the surface of the skin 1700, during a hair removal procedure, according to another embodiment of the invention. Probe 1750′ is positioned such that an active electrode 1760 is spaced a short distance from the skin 1700 adjacent to a target hair follicle 1702″. Probe 1750′ includes a return electrode 1762. In use, probe 1750′ is coupled to a high frequency power supply for applying a high frequency voltage between active electrode 1760 and return electrode 1762. An electrically conductive fluid 1770 is delivered to the distal end of probe 1750′ so as to occupy at least a portion of the gap between active electrode 1756′ and the target site. Upon application of a high frequency voltage between active electrode 1760 and return electrode 1762, a plasma is formed between active electrode 1760 and the target hair follicle 1702″. The high frequency voltage is sufficient to ablate or destroy at least a portion of the target hair follicle 1702″, without the need for direct contact between probe 1750′ and the target hair follicle 1702″. The skin 1700 may be shaved prior to the epilation process, for example, to increase visibility in the target area.

Thus, according to one aspect of the invention, the skin can be epilated in a process wherein the active electrode tip contacts only an electrically conductive fluid. In procedures of this type, the active electrode is typically spaced from the skin surface by a distance in the range of from about 0.05 mm to 5 mm. This spacing allows for the continual re-supply of electrically conductive fluid at the interface between the active electrode and the target tissue surface. This continual re-supply of the electrically conductive fluid helps to ensure that a plasma layer will remain over at least a portion of the active electrode between the active electrode and the tissue surface. It should be understood that, unlike conventional hair removal processes (e.g., electrolysis), the hair shafts do not have to be clamped, nor does the skin surface have to be penetrated in order to remove or destroy follicular tissue in the target area. Consequently, the hair removal procedures of the instant invention can be performed more quickly, and with less pain and discomfort to the patient.

FIG. 45 schematically represents a series of steps involved in a method of epilating the skin of a patient, according to the invention, wherein step 1800 involves positioning an active electrode of an electrosurgical probe in at least close proximity to a target hair follicle. The probe positioned in step 1800 may have the elements, features, and characteristics of one or more of the probes described hereinabove (e.g., with reference to FIGS, 42A-B, 43A-C, and 44B-C). In one embodiment, the probe has a solitary shaft and a solitary elongate active electrode extending distally from the shaft. Typically, the active electrode has a length in the range of from about 1.0 mm to 10.0 mm, and a width in the range of from about 0.1 mm to 2.0 mm, or less. The distal end of the shaft may be beveled (e.g., FIG. 43A), or bent (FIG. 44C), to facilitate positioning the distal end of the probe in the vicinity of the target hair follicle. The skin 1700 may be shaved prior to step 1800 to improve visualization of the target hair follicles. In addition, step 1800 may be performed with the aid of suitable magnification of the target site, as necessary.

Step 1802 involves delivering an electrically conductive fluid, such as isotonic saline, to the active electrode. The electrically conductive fluid provides a current flow path between the active electrode and a return electrode, and promotes establishment and maintenance of a plasma adjacent to the active electrode. Typically, the return electrode is disposed on the probe at a location proximal to the active electrode. The electrically conductive fluid further serves to cool tissue adjacent to the target site, thereby minimizing any thermal damage to the tissue. According to one aspect of the invention, the active electrode includes at least one groove therein, the groove(s) adapted for promoting flow of electrically conductive fluid along the active electrode towards, or beyond, the distal tip of the active electrode. In one embodiment, step 1802 may be omitted, and the method can rely on the presence of intrinsic electrically conductive fluid in the skin tissue for generation and maintenance of a plasma.

Step 1804, involves applying a high frequency voltage between the active electrode and the return electrode, wherein the applied voltage is sufficient to ablate, destroy, damage, or inactivate at least a portion of the tissue of the targeted hair follicle. In one embodiment, the voltage applied in step 1804 is sufficient to completely destroy, or irreversibly inactivate, the targeted hair follicle. Such treatment results in the effective epilation of skin, and can efficiently eliminate unwanted hair from various regions of the body. For example, using apparatus and methods of the invention, excess hair of an adult female can be permanently removed from the eyebrows, the face, or the legs. Typically, during the entire epilation process, the tissue is not exposed to a temperature in excess of about 90° C.

Other modifications and variations can be made to the disclosed embodiments without departing from the subject invention. For example, other numbers and arrangements of treatment units and fluid delivery ports on the treatment assembly are possible. Similarly, numerous other methods of treating or ablating skin tissue using electrosurgical apparatus of the invention may be apparent to the skilled artisan. In addition, apparatus, and systems of the invention may be used to treat various tissues other than the skin. Thus, while the exemplary embodiments of the present invention have been described in detail, by way of example and for clarity of understanding, a variety of changes, adaptations, and modifications is obvious to those of skill in the art. Therefore, the scope of the present invention is limited solely by the appended claims.

What is claimed is:

1. An electrosurgical apparatus for forming a plurality of graft sockets in the skin of a patient, comprising:
a handle having a working end; and
a treatment assembly disposed at the working end, wherein the treatment assembly includes a plurality of treatment units, each of the plurality of treatment units including a shaft having a shaft proximal end and a shaft distal end, an electrode support disposed on the shaft distal end, and an active electrode terminal disposed on the electrode support.

2. The apparatus of claim 1, wherein the treatment assembly is adapted for removable attachment to the handle working end.

3. The apparatus of claim 1, wherein the treatment assembly includes a base, the plurality of treatment units are affixed to the base, and the base is adapted for removable attachment to the handle working end.

4. The apparatus of claim 3, wherein each of the plurality of treatment units extends distally from the base.

5. The apparatus of claim 3, wherein the base is beveled.

6. The apparatus of claim 1, wherein the handle comprises a sterilizable housing.

7. The apparatus of claim 1, wherein the handle houses a distal connection block adapted for electrically coupling the treatment assembly to the handle working end.

8. The apparatus of claim 7, wherein the handle further houses a proximal connection block adapted for electrically coupling the handle to a high frequency power supply.

9. The apparatus of claim 1, wherein each of the plurality of treatment units includes a return electrode.

10. The apparatus of claim 1, further comprising a fluid delivery unit for delivering an electrically conductive fluid to each of the plurality of treatment units.

11. The apparatus of claim 10, wherein the fluid delivery unit comprises a plurality of fluid delivery channels.

12. The apparatus of claim 11, wherein each of the plurality of fluid delivery channels is located substantially adjacent to a corresponding one of the plurality of treatment units.

13. The apparatus of claim 11, wherein each of the plurality of fluid delivery channels terminates distally in a fluid delivery port, and the fluid delivery port is located adjacent to a distal end of one of the plurality of treatment units.

14. The apparatus of claim 10, wherein the fluid delivery unit is adapted for supplying an electrically conductive fluid to a distal end of each of the plurality of treatment units, and the electrically conductive fluid provides a current flow path between the active electrode terminal and a return electrode.

15. The apparatus of claim 1, wherein the active electrode terminal extends distally from the electrode support.

16. The apparatus of claim 1, wherein the active electrode terminal has a pointed distal end.

17. The apparatus of claim 1, wherein the active electrode terminal is conical in shape.

18. The apparatus of claim 1, wherein the treatment assembly comprises from about two to about 16 treatment units.

19. The apparatus of claim 1, wherein the plurality of treatment units are arranged in a square configuration.

20. The apparatus of claim 1, wherein the working end comprises a handle distal face.

21. The apparatus of claim 20, wherein the handle distal face is adapted for removably accommodating the treatment assembly.

22. The apparatus of claim 20, wherein the shaft of each of the plurality of treatment units extends distally from the handle distal face.

23. The apparatus of claim 20, wherein the handle distal face is substantially planar.

24. The apparatus of claim 20, wherein the handle distal face is substantially square.

25. The apparatus of claim 20, wherein the handle distal face is beveled.
